(19) United States
(12) Patent Application Publication
(10) Pub. No.: US 2025/0387196 A1
(43) Pub. Date: Dec. 25, 2025

(54) SEGMENTAL OSCILLATION TOOL AND LUBRICANT

(71) Applicant: Michael J. Florman, Garden Grove, CA (US)

(72) Inventor: Michael J. Florman, Garden Grove, CA (US)

(21) Appl. No.: 19/203,134

(22) Filed: May 8, 2025

Related U.S. Application Data
(60) Provisional application No. 63/643,929, filed on May 8, 2024.
(60) Provisional application No. 63/761,241, filed on Feb. 21, 2025.

(51) Int. Cl. A61C 1/06; A61C 1/08; A61C 3/06; A61C 3/12

(52) U.S. Cl. CPC A61C 1/06; A61C 1/082; A61C 1/087; A61C 3/06; A61C 3/12

(57) ABSTRACT

A tool for interproximal reduction includes a handpiece and a substantially solid disk-shaped member. The hand tool has a motor coupled to a coupling member. The handpiece is for creating oscillating motion in an oscillation range of 90 degrees or less. The disk-shaped member has a front face and a rear face and defines two or more segments that are different from one another. Each segment has different properties comprising one or more of perforations, cutting surfaces, and abrasives. The disk-shaped member has a centrally disposed aperture for coupling to the handpiece with the coupling member. The properties only partially cover one or more of the front or rear face of the disk. The handpiece is configured to permit a user to position their fingers directly adjacent the dish-shaped member during operation of the handpiece.

### CROSS-REFERENCE TO RELATED APPLICATION

This application claims priority to U.S. Provisional Application No. 63/643,929, filed May 8, 2024 and to U.S. Provisional Application No. 63/761,241, filed Feb. 21, 2025, the disclosures of which are incorporated herein by reference in their entirety.

### FIELD

The present invention concerns an oscillation tool and lubricant used in dental procedures including interproximal reduction. In particular, the present invention concerns a segmental oscillation tool and lubricant.

### BACKGROUND

Teeth contact each other inside the dental arch. They touch each other at a point or area in various shapes and sizes. In a simple example, two curved teeth will touch each other at a single point. This point can be as narrow as 1/100^{th} of a mm, or as wide as multiple millimeters depending on the abrasion the two teeth have experienced touching each other and rubbing against each other over time. Misaligned teeth can contact each other in places that are not the correct anatomical position of the “tooth contact” due to the position of the teeth. Some tooth contacts are “long” and others “wide,” as compared to a single point of contact.

In orthodontics, dentists in some cases need to slenderize or reduce the width of the teeth to allow all the teeth in the dental arch to fit in an anatomical “horseshoe” or “U” shape jaw bones. Obtaining access to the tooth contacts to slenderize or reduce them to make space interproximally is difficult because the practitioner needs to cut away enamel on the teeth creating the contact.

There are multiple methods that use different dental instruments to remove enamel. These include:
1) Rotary cutting instruments affixed and driven by either an electric or air driven motor connected to a contra angle dental handpiece,
2) Abrasive cutting instruments, such as strips of metal with impregnated adhered, with diamond being the most common abrasive, either natural or artificial,
3) Metal disks that rotate in a circular motion that have abrasives fused to the metal substrate,
4) Metal strips held between a “harp” that resembles a hacksaw,
5) A metal sword that reciprocates back and forth that has abrasive associated with it, and
6) A semi-circular pie shaped sanding instrument that oscillates back and forth in an arc affixed to a contra angle air or electric handpiece.

The instruments utilized are powered by electric, air motor, or hand power using the fingers to move through the dental contact to cut the enamel. For contacts that need to be only opened very small amounts, such as 0.05 mm to 0.3 mm, the procedure needs to be performed cautiously so as to not perform too much interproximal reduction. If too much enamel is removed, it will be impossible to close all the space in some instances. In today's world of orthodontics, clear aligners like Invisalign® require precise and small amounts of slenderizing to allow the aligners to move the teeth into dental arches.

There are some known metal cutting instrument coatings that may be useful in the present invention, including: Teflon, Graphite, Molybdenum Disulfide (MoS₂), Silver, Nickel-Teflon (Ni-PTFE), Copper, Zinc, and Phosphorus, among other coatings. Teflon and similar coatings can be fused to dental cutting instrument abrasives or metal cutting surfaces to enhance and lubricate the cutting instruments during interproximal reduction. Other materials can be coated to a metal substrate, like Diamonds.

Graphite coatings are often used for their lubricating properties in applications where high temperatures and heavy loads are present. Graphite provides excellent dry lubrication and can reduce friction.

Molybdenum Disulfide (MoS₂): MoS₂ coatings are another popular choice for providing lubrication to metal surfaces. Like graphite, MoS₂ offers dry lubrication properties and can withstand high temperatures and pressures, friction, and wear on metal surfaces.

Silver coatings can provide lubrication and anti-galling properties, particularly in applications where metals are subjected to sliding or rotating motions under high loads. Nickel-Teflon (Ni-PTFE) coatings combine the lubricating properties of Teflon with the corrosion resistance of nickel. These coatings are often used in applications where low friction and chemical resistance are required.

Copper coatings can provide lubrication and anti-seize properties, particularly in applications where metal parts are subjected to high temperatures and pressures. Zinc coatings, such as zinc-iron alloys, can provide sacrificial corrosion protection and lubrication properties to metal surfaces, particularly in outdoor or corrosive environments. Phosphorus-based coatings, such as electroless nickel-phosphorus or phosphate coatings, can provide lubrication and anti-wear properties to metal surfaces, particularly in applications where sliding or rolling contact occurs.

In general, the Interproximal Reduction (“IPR”) procedure is a miserable dental procedure because the dental practitioner needs to cut the teeth while avoiding problems like overcutting the enamel or cutting away too much enamel, cutting outside the space created by the two teeth surfaces touching, or causing “ledging” or iatrogenic dentistry. The present invention is directed toward making the IPR procedure less difficult, risky and more accurate.

Some examples of prior IPR devices include the K omet Segmental IPR System, Profin (Dentatus), SpaceFile (Dentsply) and other similar reciprocating saws. The K omet Segmental IPR system requires the initial use of very thin stainless steel abrasive coated strips, such as Brasseler™ strips, which need to be manually moved between teeth to open space, before the oscillating instrument can be used. The thinnest Brasseler strip is 0.08 mm. The thinnest K omet segmental instrument used for IPR is labeled the 0.2 mm instrument which has a thickness that ranges between 0.13-0.15 mm. Komet's system also has an awkward handpiece/cutting segment relationship, making the already stressful process even more stressful.

Other systems like Profin (Dentatus), SpaceFile (Dentsply), and similar linear reciprocating saws work well once contact is opened, but like Komet, they rely on contra-angle handpieces, which limits the dentist's ability to get in between the teeth's contact to open the space. Basically, you need very thin instruments of at least 0.08 mm or less to navigate past the contact and polish the space open. These systems are awkward to use in the mouth and, as a result, their acceptance has been low.

As discussed above, during the process of interproximal reduction, dentists use manual abrasive tools, like disks, swords, and strips to abrade and sand enamel structure from the interproximal surfaces between teeth. In other dental procedures where a high-speed handpiece drill bit is used, lubrication is provided by the use of water that is sprayed on the cutting surfaces of the drill bits and tooth. Because IPR uses hand instrumentation, a water source that sprays on the tooth or cutting instrument surfaces is not traditionally used. Lubrication, however, assists in completing the IPR cutting process.

### SUMMARY

A tool for interproximal reduction includes a hand tool and a substantially solid disk-shaped member. The hand tool has a motor coupled to a coupling member. The hand tool is for creating oscillating motion with a preferred degree of oscillation of 180 degrees or less. The disk-shaped member has a front face and a rear face and defines two or more segments that are different from one another. Each segment has different properties comprising one or more of perforations, cutting surfaces, and abrasives. The disk-shaped member has a centrally disposed aperture for coupling to the hand tool with the coupling member. The properties only partially cover one or more of the front or rear face of the disk. The handpiece is configured to permit a user to position their fingers directly adjacent the dish-shaped member during operation.

The properties may be a radially extending cutting member extending outwardly from the aperture to the outer circumference of the disk-shaped member. The properties may be an abrasive area and the abrasive area is spaced from the aperture. The abrasive area may be shaped as a ring of abrasive that is positioned directly adjacent an outer periphery of the disk in one or more of the segments. The properties may include one or more perforations that extend through the substantially solid disk-member. The one or more perforations may comprise a plurality of perforations arranged in a pattern, with the perforations being positioned closer to the outer periphery of the disk than to the centrally disposed aperture.

The disk may be made of plastic or metal and may have a thickness of about 0.05 mm. The degree of oscillation may be between 1 degree and 45 degrees.

In another embodiment, a cutting member for use with an interproximal reduction hand tool includes a substantially solid disk-shaped member having a front face and a rear face and defining two or more segments that are different from one another. Each segment has different properties comprising one or more of perforations, cutting surfaces, and abrasives. The disk-shaped member may have a centrally disposed aperture for coupling to the hand tool using the coupling member. The properties may only partially cover one or more of the front or rear face of the disk

One of the properties may be a radially extending cutting member extending outwardly from the aperture to outer circumference of the disk-shaped member, an abrasive area where the abrasive area is spaced from the aperture, or one or more perforations that extend through the substantially solid disk-member. The abrasive area may be shaped as a ring of abrasive that is positioned directly adjacent an outer periphery of the disk in one or more of the segments. The one or more perforations may include a plurality of perforations arranged in a pattern, with the perforations being positioned closer to the outer periphery of the disk than to the centrally disposed aperture. The disk may be made of plastic or metal and may have a thickness of about 0.08 mm.

In another embodiment, a cutting member for use with an interproximal reduction hand tool is a substantially solid wedge-shaped member forming a partial circle. The partial circle may be ¼ or less of a circle and may have a front face and a rear face, with the wedge-shaped member having different properties comprising one or more of perforations, cutting surfaces, and abrasives. The wedge-shaped member may have an aperture defined at a pointed end of the wedge-shaped member for coupling to the hand tool. The properties may only partially cover one or more of the front or rear face of the wedge-shaped member.

One of the properties may be a radially extending cutting member extending outwardly from the aperture to outer circumference of the disk-shaped member, an abrasive area and the abrasive area is spaced from the aperture, or one or more perforations that extend through the substantially solid disk-member. The abrasive area may be shaped as a ring of abrasive that is positioned directly adjacent an outer periphery of the disk in one or more of the segments. The one or more perforations may include a plurality of perforations arranged in a pattern, with the perforations being positioned closer to the outer periphery of the disk than to the centrally disposed aperture.

The member may be coated in whole or in part with a naturally lubricating surface treatment, such as Teflon or the like. The abrasive material may be fused to the cutting member and the cutting member may be coated with Teflon or other materials to enhance the abrasive ability to get in between the teeth's contacts and enhance the abrasive's efficiency.

### BRIEF DESCRIPTION OF THE DRAWINGS

FIG. 1 is a plan view of an example IPR disk shape according to the invention;

FIG. 2A is a perspective view an example IPR disk shape according to the invention;

FIG. 2B is an exploded view of an IPR disk similar to that shown in FIG. 2A;

FIG. 3 is a plan view of another example IPR disk shape according to the invention;

FIG. 4 is an exploded view of another IPR disk shape showing a single segment of the disk;

FIG. 5A is a plan view of an alternative example IPR disk shape according to the invention;

FIG. 5B is a perspective view of a cutting edge that may be used with the IPR disk according to the invention;

FIG. 6 is a plan view of an alternative example IPR disk shape according to the invention;

FIG. 7 is a plan view of an alternative example IPR disk shape according to the invention;

FIG. 8 is a plan view of another alternative example IPR disk shape according to the invention;

FIG. 9 is a perspective view of another example IPR disk shape according to the invention;

FIG. 10 is a schematic showing different configurations for a round disk having four segments;

FIG. 11 is a plan view of a front side of an example IPR disk shape according to the invention;

FIG. 12 is a plan view of a rear side of the disk of FIG. 11;

FIG. 13 is a representation of a cross-sectional view of the disk of FIG. 11;

FIG. 14 is a perspective view of a wedge-shaped member that incorporates features of the disk shown in FIG. 11;

FIG. 15 is a perspective view of an alternative wedge-shaped member that incorporates features of the disk shown in FIG. 11;

FIG. 16 is a perspective view of an alternative wedge-shaped member that incorporates features of the disk shown in FIG. 11;

FIG. 17 is a perspective view of an alternative wedge-shaped member that incorporates features of the disk shown in FIG. 11;

FIG. 18 is a front view of an example handle according to another aspect of the invention incorporating a stationary cutting blade in the form of a sanding strip;

FIG. 19 is a front view of another example handle similar to that shown in FIG. 18 incorporating a stationary cutting blade in the form of a sanding half circle;

FIG. 20 is a front view of another example handle similar to that shown in FIG. 18 incorporating a rotating circular cutting blade;

FIG. 21 is a front view of another example handle similar to that shown in FIG. 18 incorporating a rotating circular cutting blade;

FIG. 22 is a side view of the handle shown in FIGS. 20-21 incorporating a set screw within the handle for setting the position of the cutting blade;

FIG. 23 is a view of the handle shown in FIGS. 20-21 incorporating a set screw extending inwardly through the handle to the cutting blade to set the position of the cutting blade;

FIG. 24 is a front plan view of a disk according to the invention having a surface treatment adjacent the outer periphery of the disk;

FIG. 25 is a front plan view of an alternative embodiment of the disk showing an additional surface treatment to delineate the segments from one another;

FIG. 26 is a front plan view of an alternative embodiment of the disk showing an additional structure that is attached to the surface of the disk for stabilizing the disk;

FIG. 27 is a cross-sectional side view of the disk of FIG. 26; and

FIG. 28 is a perspective view of an example handpiece with an attached disk according to the invention.

### DETAILED DESCRIPTION

The present invention is directed to a new method of interproximal reduction (“IPR”) in dentistry that involves very high torque, segmental oscillation, and an angle of rotation between 180 degrees and 1 degree. A preferred degree of oscillation is between 3 degrees and 6 degrees. The present invention is also directed toward a lubricant that can be used in IPR procedures as well as other procedures.

The present invention is directed toward an IPR dental tool that eliminates the need for manual strips, spinning disks, and linear reciprocating tools. It uses an oscillating segmented metallic or plastic disk with lubricant to reduce friction and optionally with fluoride to strengthen enamel. This provides for a fast and safe technique for interproximal reduction. Disks may be available in a variety of thicknesses, such as about 0.1 mm, about 0.2 mm, about 0.3 mm, and about 0.4 mm thick, among other thicknesses below, above, or in between. These measurements apply to the thickest (double-sided) segment of each disk. The single-sided segment of the thinnest disk (about 0.1 mm) will be thinner (approximately 0.08 mm) because it does not have abrasive on both sides of the disk. It only has abrasive on one side of the disk. The 0.08 mm thickness is the same size as the thinnest Brasseler Yellow strip. The invention further describes a segmented disk that offers a thinner thickness ranging from about 0.04 mm to about 0.07 mm.

IPR Disks are typically round, although they could be other shapes since they are designed to oscillate rather than spin. The present invention can alternatively be used with wedge-shaped sections, which also cut by oscillating, as will be described in greater detail below.

The IPR device includes a handpiece (not shown) that is driven by an air motor or an electric motor. An IPR disk or wedge is then attached to an endpiece of the handpiece for operation on a patient's teeth. The endpiece may be a mandrel or other attachment device. The handpiece has a straight or contra angle. The disk is a round metal that is impregnated with abrasive. The handpiece operates by oscillating the disk back and forth and this back and forth motion serves to slowly polish down the tooth surfaces that are adjacent the abrasive side of the disk (or wedge). The metal disk of the present invention, when oscillating, does not have enough circular momentum force to cut the doctor or patient's soft tissue. In contrast, prior art devices that utilized a spinning metal disk rotated at speeds of up to 40,000 RPM. Because the disk oscillates instead of spinning, it alleviates risks of injury to a patient's hard and soft tissue, and dental operators associated with a spinning disk.

In use, the disk is wedged between a patient's teeth either stationary or oscillating, using force from the dentist's hand pushing the disk down which splays the teeth apart so the thin metal disk can be positioned between the teeth. The thin metal disk can range in thickness from about 0.04 mm to about 2.0 mm. The disk has cutting surfaces, such as diamond coated cutting surfaces, or abrasive sections which are positioned to contact one or both sides of the splayed teeth.

The IPR disk is divided into segments, with each segment having a different feature on one or both sides of the disk. The disks shown herein are round or wedge-shaped. However, other shapes can alternatively be used, such as circular, triangular, square, rectangular, polygonal, or other shapes. The disk can be segmented in multiple segments in multiple directions.

The disk may be segmented in multiple segments in multiple directions. When the disk is circular, a section of the circle may be pie-shaped and have sides that extend along two separated radii. A portion of the circumference of the circular disk, also known as an arc of the circle and the 2 radii of the circle meet at both endpoints of the arc forming the sector or section. The shape of the sector of the circle looks like a pizza slice or a slice of pie. The number of sectors on the disk can range from more than one up to ten, with a more specific range of 2-6, and a more ideal range of 3-4. The circular instrument can have a diameter ranging from about 5 mm to about 35 mm, with an ideal diameter of 22 mm.

The disk is typically flat and has two faces, including an upper face and a lower face. When the disk is mounted on a mandrel or shank, it allows the disk to move. When the disk is mounted on the mandrel or other attachment member of the handpiece, there is an “up” side and a “down” side. The “Up” or “Down” sides of the disk are defined relative to the direction of the disk/mandrel as its placed on the handpiece or shank of the handpiece. The handpiece is connected to a motor, which is either electric or air driven, and the handpiece rotates the mandrel and disk.

After the disk is wedged between the teeth, the motor of the handpiece can be turned on to activate the disk to oscillate. Because the diamond cutting abrasive is apical to the contact, the friction between the mesial and distal tooth surfaces against the metal disk is reduced as compared to if the abrasive coating was wedged between the tooth surfaces. If the disk cannot be wedged between the teeth's contact, lubricant can be applied to help facilitate the insertion, along with activating the motor to oscillate the disk.

As the oscillation commences, the dentist moves the disk occlusal-apically and pulls the abrasive part of the disk through the teeth contact. This sands away the enamel safely in either one of the adjacent tooth surfaces or both of the adjacent tooth surfaces, depending upon which side of the disk is positioned adjacent the tooth surface. If the side of the disk that has an abrasive surface is positioned on one side of the disk, only one tooth will be sanded. If both sides of the disk have an abrasive surface, then both teeth will be sanded.

Friction between the two tooth surfaces during the IPR procedure can be reduced using a lubricant. The lubricant may be water spray. The lubricant may be an oral therapeutic lubricant fabricated from cosmetic ingredients that are used to lubricate or retain moisture. The lubricant may include abrasives, a polishing agent, fluoride, or other enamel remineralization chemicals. Alternatively, the lubricant does not need to include any additional additives. Examples of different formulations for lubricants are discussed below.

Friction between the two tooth surfaces can also be reduced during the IPR procedure based upon the features of the disk. The disk may have abrasive fused to the outer most edge of the disk. In one embodiment, the disk has the abrasive fused on the outer most edge of the circle, approximating less than 30% of the total radius. One possible range for the location of abrasive on the disk is in a range of between 2 and 15% of the radius, with the abrasive being positioned adjacent an outer edge of the disk. The abrasive may be spaced from the edge of the disk or could be positioned directly at the edge and extend inwardly from the edge of the disk.

The disk can be perforated with many holes in a honeycomb pattern. Alternatively, fewer perforations may be provided. When high torque is applied to the disk during oscillation as the teeth push against the disk, more perforations decrease the friction between the teeth and the disk while fewer perforations provide for more solid metal along with any abrasive coatings to contact the teeth's perforations, which is more effective at polishing or sanding.

The disk can oscillate at a degree that ranges from about 1 degree to about 270 degrees. A more ideal range is about 1 degree to about 200 degrees, another preferred range is about 10 degrees to about 45 degrees, and another oscillation range is about 0 to 90 degrees, about 0 to 180 degrees, about 1-10 degrees, and about 2-6 degrees.

The oscillation can be at high speed up to 40,000 RPM, with a small degree of forward and reverse oscillation ranging from 1 degree to 50 degrees.

The motor can be powered by an air or electric motor.

The range of oscillation can be set or adjusted to correlate to a segment on the disk. An example of this is that if the disk is divided in quarters, each segment is 90 degrees. An electric motor that has the ability to have a program create more than one oscillation frequency so that the dentist can switch between various oscillations. An air driven motor will need to be designed and geared to perform a fixed oscillation, so if the user wishes to have more than one oscillation, he/she will need multiple geared handpieces. An oscillating hand piece can be sold and designed to offer fixed degrees of oscillation ranging from about 1 degree to about 180 degrees. The lower the degree of oscillation, the more control the dentist will have. For example, the Komet™ system uses a 15 to 30 degree oscillation where a more ideal degree of oscillation is 1 to 6 degrees. The lower the degrees, the more of a vibration effect is achieved.

The disk must be able to oscillate at a high Torque so it can start from a complete stop and begin moving as it is pinched between two teeth pushing against it. A range or torque can include 0.4 N cm to 6 N cm, with a preferred torque level of about 3. This torque level is dependent upon the motor driving the handpiece, the gear ratio of the handpiece, the tool utilized, the tightness of the disk between the teeth, and the thickness of the disk, among other factors.

Different types of energy may be used to operate the cutting mechanism. Oscillation may be used through an oscillation arc. Alternatively, ultrasonic energy may be used, if desired. Ultrasonic energy includes magnetostrictive ultrasonic scalers (magneto) or Piezoelectric ultrasonic scalers (piezo). Magnetostrictive ultrasonic scalers use a stack of metal strips or a metal rod that expands and contracts in a magnetic field. The motion is typically elliptical or circular. The frequency is typically in a range of 18,000-45,000 Hz. Water is used to cool both the tip and the metal stack. Piezoelectric Ultrasonic scalers use crystals (usually quartz or ceramic) that expand and contract under electrical current. The tip motion is typically linear (back and forth). The frequency is typically 25,000-50,000 Hz. Water is primarily used to cool the tip and for lavage.

In use, the disk is mounted to a shank or mandrel and placed on an oscillating dental hand piece into its chuck. The disk is pushed through the contact between two teeth that are touching one another, either entering from the occlusal or incisal surface. Before insertion between the teeth, lubrication is applied to the disk and the disk carries the lubrication between the two teeth at the point of contact, or the lubricant is applied to the teeth at the teeth's contacts, and the disk pushes it into the contact. Once the disk is wedged between the teeth, the motor on the hand piece is activated, and the disk begins to oscillate. As the oscillation occurs, it sands or polishes down the enamel between the teeth to create a space between the teeth.

The disk can be coated, plated, or covered with a varying range of abrasives containing different particle sizes or hardnesses with varying thicknesses. A disk with four (4) segments will have eight (8) surfaces, four (4) “up” surfaces and four (4) “down” surfaces. Any combination of coatings on the eight (8) segments can exist or be purposely not coated creating a single sided coated segment, or coated on both sides creating a segment with coating on both sides of the disk. All coatings on the disk may use the same abrasive material in thickness, particle size, density and the like, creating segments with different thicknesses based on the disk having a single side that is coated, either “up” or “down.” One segment may have a double-sided coated segment, e.g., “up” and “down.” The thicknesses of the double-sided segment will be thicker than either of the single sided segments. Coatings on the disk can vary in thickness, density, and particle size, creating a disk where all the segments have an equal thickness, regardless of whether the segment is coated on one side or both sides.

Disk segment thickness can be altered by stamping or casting protrusions into the single thickness disk, allowing, for example, a 0.05 mm thick metal disk to have protrusions that can range from about 0.05 mm up to about 0.5 mm, depending on the elasticity of the disk metal. Abrasive material can be positioned on the disk at varying levels of the disk and can be segmented rather than in sector segments but in circumferential segments varying from the most outer circumference which is the largest circumference moving towards the center of the circle, which will have the smallest circumference.

To allow access to the contact where two teeth touch, the disk can have variations of coatings. The coatings may start, for example, at a point on the disk that is furthest from the center of the disk that is not coated. The disk is thinner where it is not coated and may include perforations in the metal. The coating extends up towards the center of the disk. The thickness of the disk can be varied by placing perforations at the leading edge. An abrasive can be either mesial or distal to the leading edge that contains no abrasive.

The full circle design of the disk allows the sanding surfaces to rotate 360 degrees allowing the user to access varying portions of the circle. The starting point in this varying thickness disk design may have no abrasive on it, or a minimal amount, and just consist of the underlying metal of the disk. The “pie” shaped sections can be treated with abrasive on one side or both sides. The “pie” shaped sections can also be coated with abrasives in varying thickness to allow four tools to be on a single disk (with the disk divided into 4 quadrants). The user need only turn the disk from a segment of the pie to change the tool from, for example, 0.1 mm to 0.2 mm to 0.3 mm to 0.4 mm, etc. The segments of the “pie” shaped sections can be divided by a noncoated section of the disk that divides the varying abrasive thicknesses, or a line can be fabricated using the abrasive. The segments of the “pie” shaped sections can be labeled using sprayed on or stamped on measurements or associated colors indicating the thickness. The varying thickness of the disk can also be graduated, where the disk begins at a small thickness such as about 0.05 mm and gradually increases along the 365-degree disk to a thickness of about 0.5 mm.

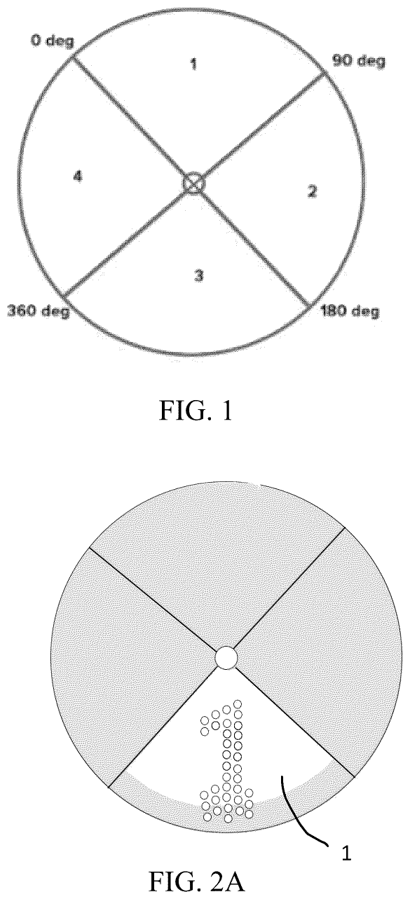

Referring to the drawings, FIG. 1 shows a disk having four sectors or segments. The disk is evenly divided into the four segments so that each segment takes up approximately 25% of the surface of the disk. The disk has an aperture in the center thereof for receiving an appendage from the hand piece, such as a mandrel or shank, onto which the disk can be attached. The segments are labeled 1-4. Each segment can have a different design to facilitate different sanding procedures. While not shown, there may be surface treatments associated with the disk surface to aid in lubrication and to facilitate polishing or sanding. As discussed above, the disk is designed to be used on a segment-by-segment basis via oscillation, so that only one segment of the disk polishes or sands at a time and the segments are not used simultaneously. The disk has a base thickness and then any coatings or abrasives that are applied to the surface will increase the thickness at the area of application. The final desired IPR space to be created will include the thickness of any abrasive coating on the disk surface.

FIG. 1 shows four separate sections on the disk, labeled 1-4. Different numbers of sections could be used, such as three (3) sections, where the disk surface is divided into thirds, or two (2) sections, where the disk surface is divided into halves. Even where the disk surface is divided into multiple sections, it may be designed so that only one of the four sections are used, or two of the four sections are used while the remaining sections are not used, for example. It is not required that all surfaces have abrasives or other surface treatments.

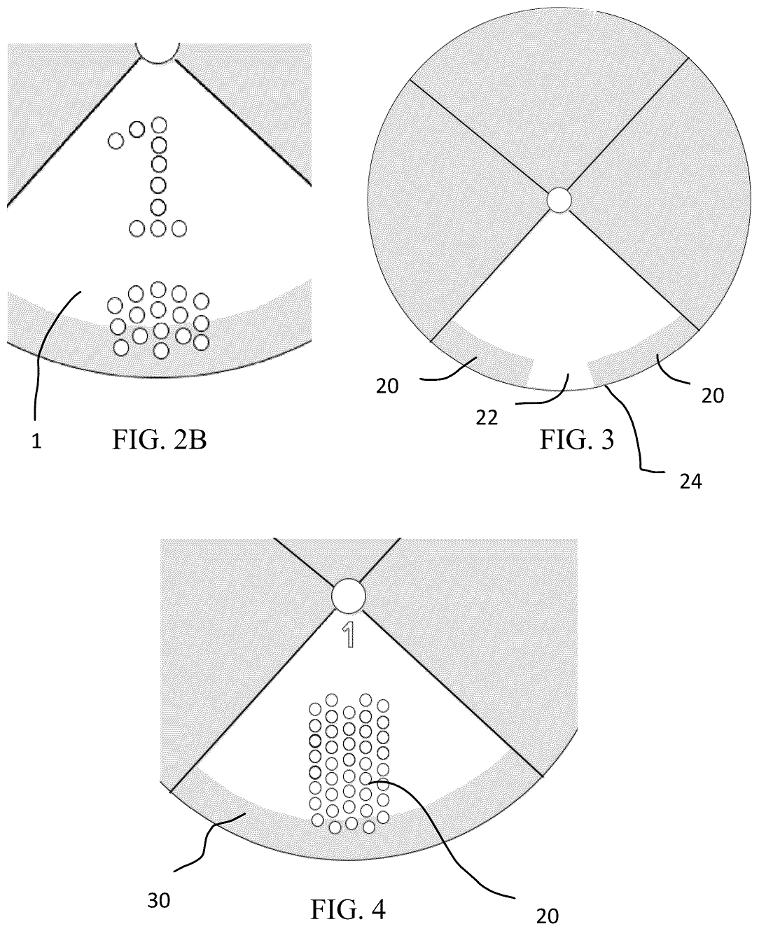

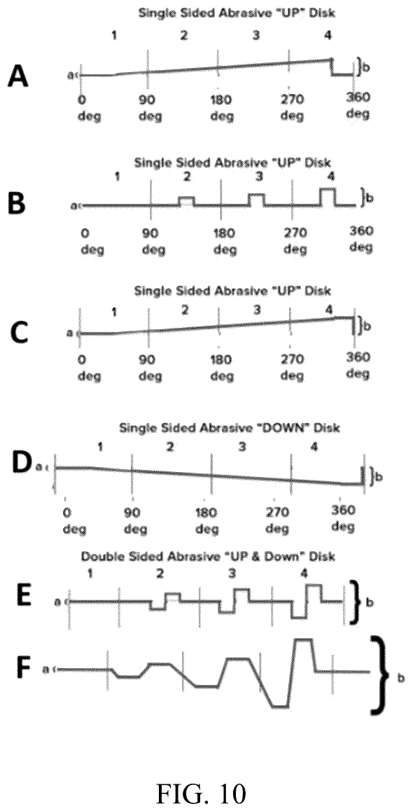

FIG. 2A shows a disk similar to that shown in FIG. 1 that is divided into quarters. The disk has an area of abrasive applied to an outer portion of the face of the disk with the remainder of the disk as shown in Section 1. As shown, the abrasive covers approximately ⅕ of the radius of the disk. In addition, perforations or holes are formed through the disk and are arranged in relatively close proximity to one another. The holes can be used to carry lubrication to the tooth surfaces and reduce the friction by having the perforations decrease the total surface area of the metal being used to perform IPR, while still offering an instrument that is structurally strong preventing the tool from bending or deforming. FIG. 2A shows the apertures forming the shape of number 1, which can represent the disk's number in a series of disks, or the disk's thickness, e.g., a number 1 representing 0.1 mm. This type of shape may be used to signify which disk to use first. Disk 1, for example, may be the thinnest disk while later disks are slightly thicker for deeper polishing. The remainder of disk 2A may have a surface treatment such as a varying thickness abrasive (referring to FIG. 10, Row C).

FIG. 2B depicts a disk similar to the disk shown in FIG. 2A, but the number 1 is positioned closer to the center of the disk while a cluster of perforations are positioned closer to the outer circumference of the disk. In this embodiment, it's possible to have the number 1 formed from fewer perforations, which can alter the stiffness of the disk. By using fewer holes to depict the identifying number, the disk can be made stiffer. By adding more holes, the disk can be made more flexible. FIG. 2B also depicts an abrasive positioned on approximately the outer ⅕ of the circumference of the disk in segment 1. In either FIG. 2A or 2B, the abrasive could be on both the front and back surfaces of the disk or could be on only one side of the disk.

FIG. 3 depicts a disk according to the invention where the disk utilizes an intermittent abrasive 20 on the outer circumference 22 of the disk. As with prior embodiments, the abrasive is shown covering part of the outer ⅕ of radius R of the disk. In this embodiment, the circumference within the segment at issue is not entirely covered with abrasive material. A space or gap 22 that is free of abrasive material is provided between each end of abrasive material at the circumference. The area 22 on the edge of the disk that is clear of abrasive allows for easier insertion into the tight dental contact. Then when the disk begins oscillating, the abrasive material 24 begins to polish and remove material to open the space between the teeth. The abrasive material could take up more of the disk, such as ⅖ ½, ⅗, or the like. The abrasive material could be positioned on one side of the disk or on both sides of the disk, depending upon whether you wish to sand one tooth or both adjacent teeth. If desired, the abrasive could be fused to the entire surface.

FIG. 4 is an alternative embodiment of the disk of FIG. 2B and includes approximately the same amount of abrasive as shown in FIG. 2B. FIG. 4 depicts a cluster of apertures 30 positioned in the cutting section in a somewhat rectangular shape. In addition, FIG. 4 shows the number 1 positioned near the center aperture of the disk. As with prior embodiments, the apertures can be used to carry lubricant and other materials to the space between the teeth. In addition, the perforations are positioned to allow less friction between the teeth contacts after the disk is slipped in between the teeth contacts. The perforations do not extend all the way to the edges of the segment to maintain the disk's rigidity during high torque oscillation.

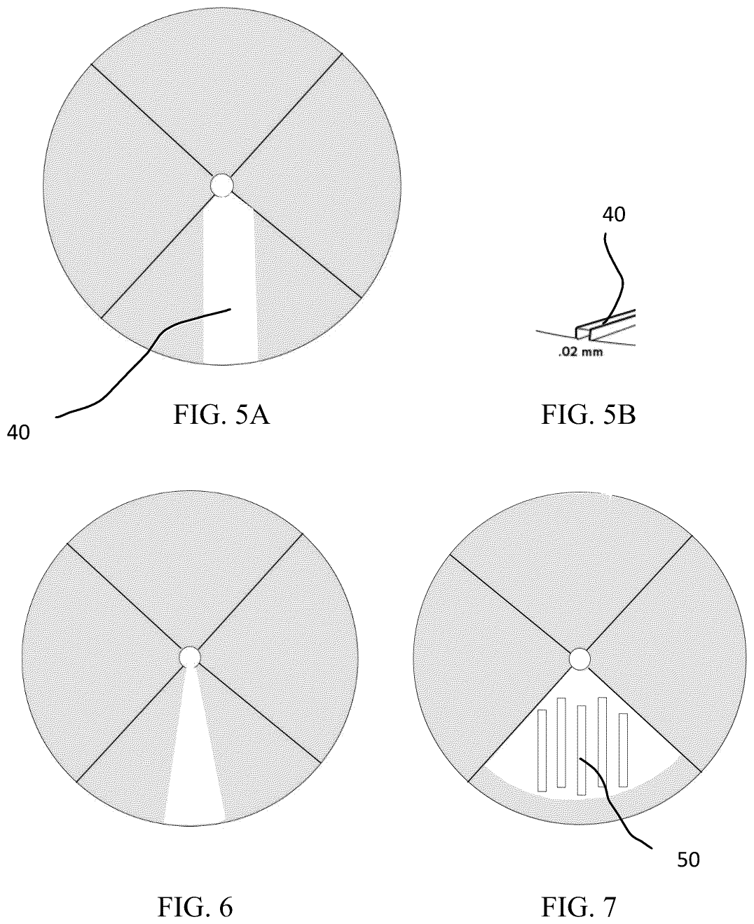

FIG. 5A depicts an alternative disk showing one segment set up for cutting, as depicted by the highlighted area. FIG. 5B depicts a perspective view of a raised area 40 having a height of 0.02 mm. This raised area may be a casting or a stamping and is used as the cutting implement for sanding or polishing an adjacent tooth surface and is positioned in the highlighted area shown in FIG. 5A. The highlighted area of FIG. 5B is substantially rectangular and can taper from the outer edge of the circumference towards the center of the disk.

In contrast, FIG. 6 depicts a similar highlighted area in which a raised area 40 may be present where the highlighted area and raised area is pie-shaped, or triangular. In both cases, the raised area emanates from the aperture in the center of the disk. Alternatively, although not show, the raised area could be present only in a limited area of the disk, such as in the outer half or outer ⅕^{th} of the radius of the disk. The raised area could have any number of different cross-sectional shapes, including rectangular, triangular, rhombus, or other shapes.

FIG. 7 depicts an alternative disk having a segment with perforations 50 that are linear and may be arranged in either a horizontal or vertical direction. FIG. 7 depicts the perforations being arranged vertically such that at least one of the perforations aligns with the radius of the disk and the other perforations are arranged substantially parallel to the radially aligned perforation. Alternatively, although not shown, the perforations could be arranged perpendicular to the radius of the disk. Alternatively, the perforations may be arranged radially, with each perforation being aligned with a radius of the disk. While linear lines of perforations are shown, the perforations could alternatively be a plurality of circular perforations arranged in a line or other shapes.

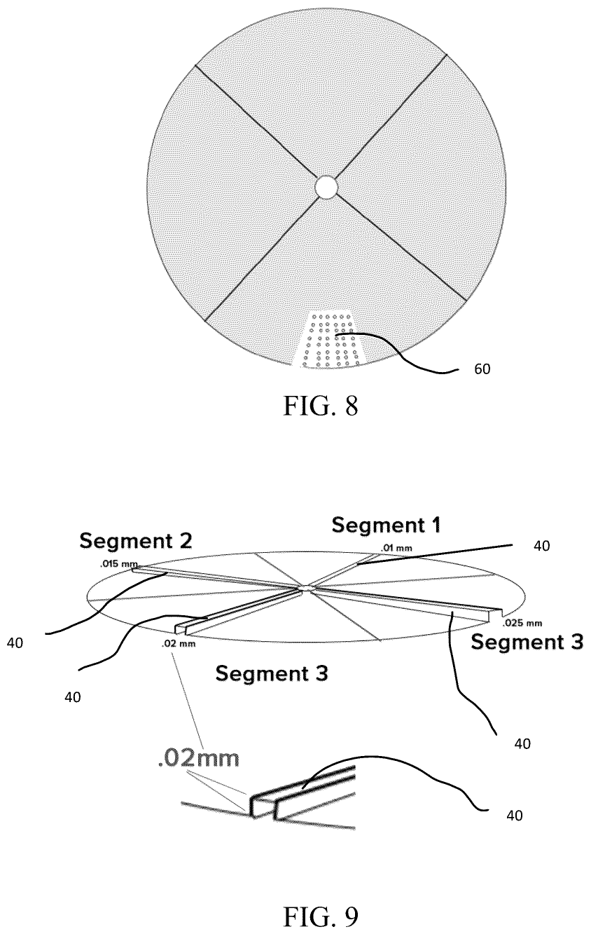

FIG. 8 depicts an alternative embodiment of the disk having four segments, showing perforations 60 at the bottom, center edge of one of the segments. In this embodiment, there is a plurality of micro or smaller holes that are positioned in the outer quarter of the wedge-shaped portion. The holes are arranged in radially extending lines. The perforations/holes are used for accepting a lubricant, among other possible uses.

FIG. 9 depicts a disk having four segments, where each segment includes a cutting surface 40 used for grinding the tooth surface. Each cutting surface radiates from the center of the disk radially outwardly to the outer edge of the disk. Each cutting surface has a substantially rectangular cross-section that is wider near the outer edge of the disk and narrower at the center of the disk.

The rectangular cross-section may be open along the bottom surface of the disk, particularly where the disk is stamped. As shown, each segment has a cutting surface with a different height. The shortest cutting surface is about 0.01 mm high, as shown in segment 1. The second highest cutting surface is about 0.015 mm, as shown in segment 2. The third highest cutting surface is about 0.02 mm, as shown in segment 3. The greatest height cutting surface is about 0.025 mm, as shown in segment 4. Each segment can be used to create a gap between two teeth. FIG. 9 also shows an exploded view of a cross-sectional shape of the rectangular raised cutting surface.

Referring to FIG. 10, the lines represent the segments of the disk representing the segments divided into a range of degrees of a circle. FIG. 10 should be viewed along with FIG. 1 and FIG. 9. FIG. 10 represents various cutting configurations for a disk having four sections, such as shown in FIGS. 1 and 9. FIG. 10, Row A, depicts a single-sided abrasive “Up” disk having the thinnest amount of abrasive material positioned on the “Up” side of the disk in section 1 (between 0 and 90 degrees). Section 2 (between 90 degrees and 180 degrees) has a thicker amount of abrasive material than Section 1. Section 3 (between 180 degrees and 270 degrees) has a thicker amount of abrasive material than Section 2, and Section 4 (between 270 degrees and 360 degrees) has half of the width of the section fused with a thicker amount of abrasive material than Section 3 and the remainder of the Section has lesser abrasive material like that of Section 1.

FIG. 10, Row B, depicts a single-sided abrasive “Up” disk where the cutting members are raised areas that provide the cutting surface. The raised areas are presented in the center of each section. Section 1 does not have a raised area. Section 2 has a raised cutting area. Section 3 has a raised cutting area that is greater than Section 2, and Section 4 has a raised cutting area that is greater than Section 3.

FIG. 10, Row C, depicts a single-sided abrasive “Up” disk where each section has a progressively greater thickness of abrasive. The abrasive drops back from the thickest amount to the least amount between Sections 4 and 1.

FIG. 10, Row D, depicts a single-sided abrasive “Down” disk where Section 1 includes thinnest amount of abrasive material. Section 2 includes a thicker amount of abrasive material than Section 1. Section 3 includes a thicker amount of abrasive material than Section 2, and Section 4 includes a thicker amount of abrasive material than Section 3. The thickness transitions from greatest to lowest thickness between Sections 1 and 4.

FIG. 10, Row E, depicts a double-sided abrasive “Up” and “Down” disk. Section 1 shows either no abrasive material or a thin coating of abrasive material. Section 2 shows opposed raised sections (one extending outwardly from the surface on each side of the disk) that have a greater height than Section 1, but lower height than Section 2. The two opposed raised areas have the same height as one another, although could have different heights if desired. Section 3 shows opposed raised sections that have a greater height than Section 2. Section 3 shows opposed raised sections that have a greater height than Section 3.

FIG. 10, Row F, depicts a double-sided abrasive “Up” and “Down” disk that has raised sections for cutting. The raised sections each have a sloped transition from a base level to the maximum level. Section 1 does not have a raised area but may have a light coating of abrasive material. Section 2 has two opposed raised areas (one facing upwardly and one facing downwardly). The two opposed raised areas have the same height as one another, although they could have different heights if desired. Section 3 has two opposed raised areas with a height of the cutting surface that is greater than the height of the cutting surfaces in Section 2. Section 4 has two opposed raised areas with a height of the cutting surface that is greater than the height of the cutting surfaces in Section 3. Section 4 represents a transition to the lowest height present in Section 1. This transition occurs after the raised cutting surface in Section 4.

As is evident, any number of different combinations of cutting surfaces may be provided. Cutting surfaces may be provided by the use of abrasives or by raised cutting surfaces. Combinations of abrasives and raised cutting surfaces may be used together or separately, as desired.

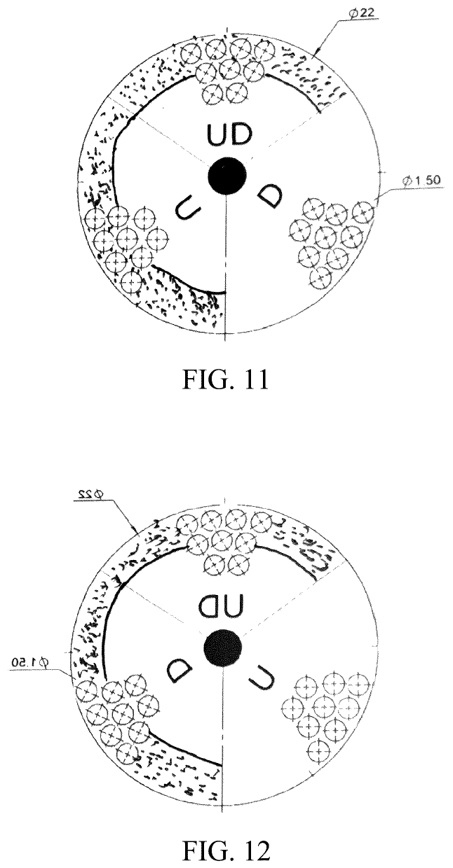

FIGS. 11 and 12 depict an alternative embodiment of the IPR disk showing a total of three (3) sections on a single disk. FIG. 11 shows the front side of the disk while FIG. 12 depicts the rear side of the same disk. In this embodiment, the disk is split into thirds so that each Section is 33.3% of the total circumference of the disk. Lines may be etched, painted, or otherwise applied to the surface of the disk to delineate the sections, if desired. In addition, markings may be etched, painted, or otherwise applied to provide identifiers for each section. As shown in FIGS. 11 and 12, there are three labeled sections, including Section U (for “Up”), Section D (for “Down”), and Section UD (for “Up” and “Down”). Section U includes abrasives or cutting surfaces on an upper side only of the disk. Section D includes abrasives or cutting surfaces on a lower side only of the disk. Section UD includes abrasives and/or cutting surfaces on both the upper and lower sides of the disk. This permits a professional to select which tooth to grind at a given time simply by rotating the appropriate section into the space between the teeth.

FIGS. 11 and 12 depict the use of an abrasive that is positioned around the outer circumference of the disk, taking up approximately ¼ to ⅕ of the surface of the disk at the outer circumference thereof. The disk also includes a plurality of holes arranged in a regular pattern, such as the triangular pattern shown. The pluralities of holes are perforations that extend through the disk. The holes can be used for receiving a lubricant to assist in inserting between and cutting the tooth. Alternatively, the holes can provide a disk with less surface area, which may also permit easier insertion and use of the disk to grind or polish the adjacent teeth. The holes are positioned adjacent to the outer circumference of the disk, but do not extend all the way to the outer circumference. The holes are positioned close together and may have a diameter of approximately 1.5 mm. Other sizes and shapes for the perforations may alternatively be used. The perforations are positioned in the center of each section near the outer periphery of the disk. The perforations are arranged to extend inwardly on the disk about half the radius thereof. The abrasive material is positioned on the U and UD sections of the disk on the front side of the disk (Shown in FIG. 11) and is positioned on the D and UD sections of the disk on the rear side of the disk (Shown in FIG. 12). A central hole is provided in the disk for attachment to a mandrel or other attachment mechanism associated with a hand tool. The segments can have abrasive that extends onto the metal between perforations or the abrasive can not be applied to the metal between the perforations.

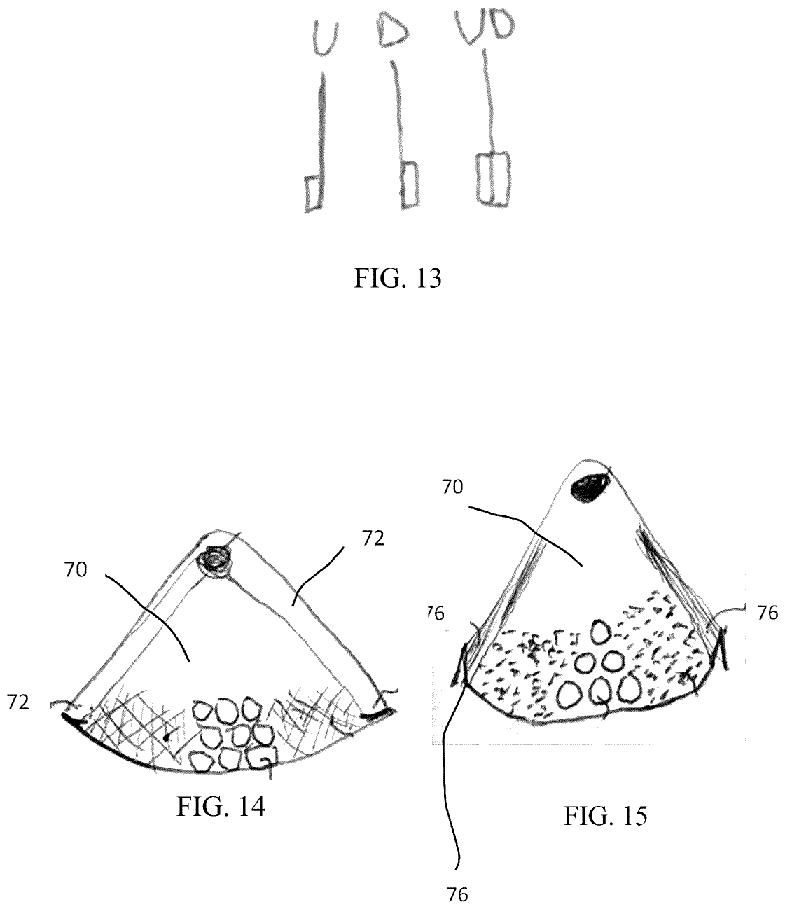

FIG. 13 depicts a cross-sectional view/edge view of the disks shown in FIGS. 11 and 12. As shown in FIG. 13, the disk has a consistent thickness and the abrasive material, increases the thickness of the disk adjacent the outer periphery thereof. The abrasive material on the U, D, and UD sides is shown as being of the same thickness. Alternatively, the abrasive material could have different thicknesses or varied thicknesses along a single section thereof.

The disks are designed so that only a single section is used at a time. Because the disk oscillates, it only permits partial rotation of the disk so that a single section engages the teeth at one time. To move to a different section, the disk is rotated so that the new section is positioned between the teeth.

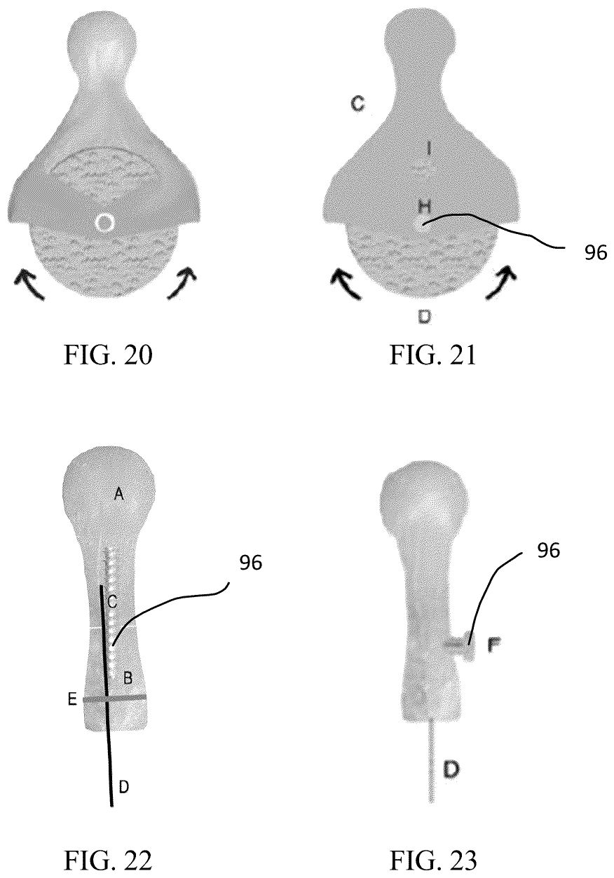

FIGS. 14-23 depict an alternative embodiment of the invention where a wedge-shape member 70 is used instead of a circular disk. Since only a portion of the disk is used at a time because of the oscillation, a wedge-shaped member 70 works equally well. The embodiments depicted in FIGS. 14-23 show different ways to assemble or fabricate the wedges so that they are finished at the edges. In each embodiment, the abrasives and perforations are substantially the same as those shown in FIGS. 11-13. Any other configuration may alternatively be used for the abrasives and/or perforations.

FIG. 14 depicts a first embodiment of a wedge-shaped member where the sides 72 of the wedge are folded over the upper surface of the wedge. In this embodiment, all sides of the wedge except the arc-shaped outer circumference have folded over surfaces. The folded over surfaces also encircle the opening in the wedge that is used to attach the wedge to a handpiece. Unlike the Komet™ segmental instruments, the folded surfaces increase the structural strength of the segment, which prevents the instrument from deforming.

FIG. 15 depicts a similar disk where the sides are folded downwardly to form an upside-down V-shape 76 along the side walls of the wedge. The opening for receiving the oscillating tool at the smaller end of the wedge shape may be surrounded by the folded over V-shaped material or the opening for receiving the oscillating tool may not be surrounded by the V-shape.

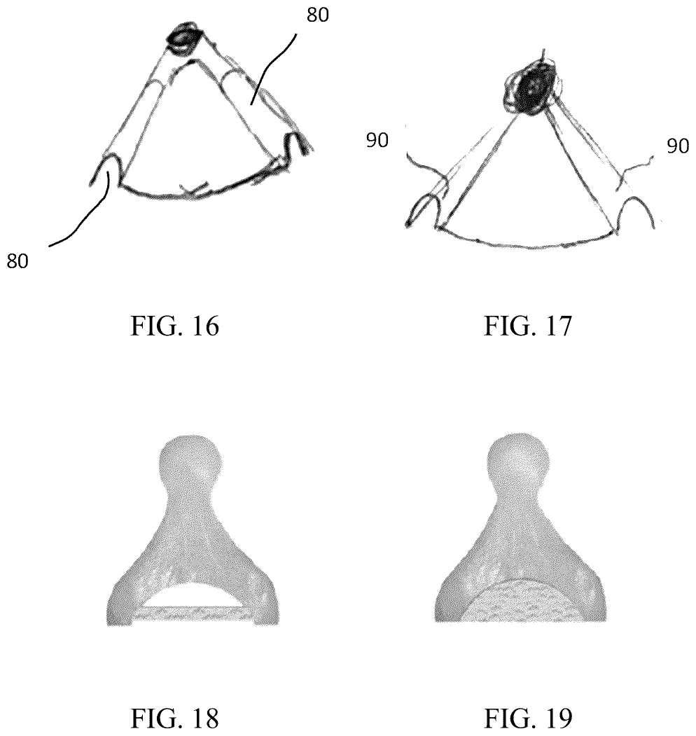

FIG. 16 depicts a wedge-shaped member where the side edges form a U-shaped channel 80 around the periphery of the side edges, while the outer circumference of the wedge is not treated. In this embodiment, the U-Shaped channel surrounds the sides and the hole for receiving the oscillation tool is also surrounded by the channel.

FIG. 17 depicts an embodiment similar to FIG. 16 that includes a U-shaped channel 90, but the u-shaped channel dissipates as it gets near the opening for receiving the oscillation tool so that the channel does not surround the opening.

The oscillation arc for this wedge system may range from about 0.05 degrees to 8 degrees, with an ideal arc of 3 to 6 degrees. The smaller the arc, the greater the potential for vibration. Because this embodiment is only a segment of a circle, the wedge is not as rigid as a full disk.

There are different methods of increasing the thickness of a disk within segments. One such method is to provide different thickness base disk metals regardless of whether the disk is up, down, or double-sided. Another method is to provide bends or divots to increase the thickness of a disk within the respective segments so that one disk can have multiple thicknesses. Another method is to use different thicknesses or sizes of abrasives to increase or decrease the thickness of the disk. It was not previously known to provide changes in height of cutting surfaces on the face of a disk or wedge by having raised areas. Moreover, prior systems for cutting in different fields use a disk having a serrated edge. The present invention does not require the use of a serrated edge. A serrated edge would be more likely to cause injury to the surrounding soft and hard tissues.

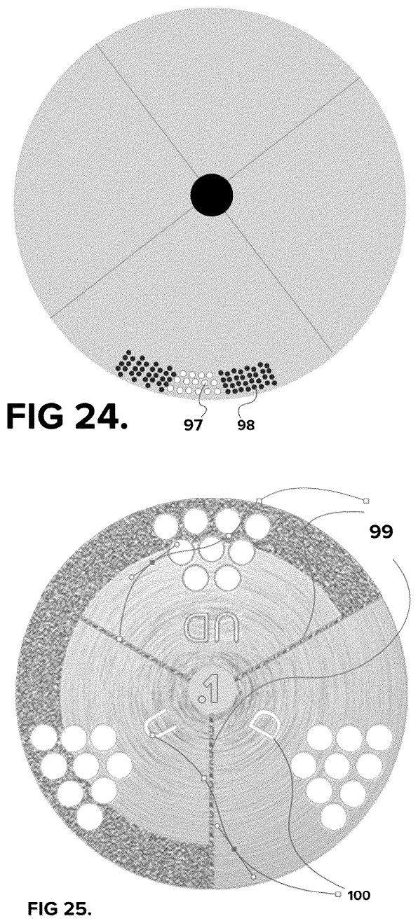

FIGS. 24-26 depict alternative embodiments of the disks. In FIG. 24, a combination of perforations 97 and dimple 98 are positioned near the outer diameter of the disk adjacent the outer periphery thereof. The perforations 97 are positioned centrally and the dimples 98 are positioned on either side of the perforations. The dimples 98 are presented on the surface of the disk and can be used for cutting and polishing purposes. The dimples 98 are either punched into the disk material, which bends the material, particularly when metal is used, or cast into the disk material, depending upon how the disk is fabricated. The combination of dimples and perforations is presented in approximately the outer ¼ to ⅕ of the radius of the disk and takes up less than the full width of the outer periphery, although they could take up more or less than is presently shown. The perforations 97 are for receiving a lubricant, among other purposes.

FIG. 25 depicts a disk that includes an abrasive coating 99 used to mark the disks. The abrasive coating is used to separate the segments or to indicate a letter or other symbol. This additional abrasive coating 99 may be the same thickness as the abrasive coating that is provided around the outer perimeter of the disk. Additional perforations 100 may be used to create letters, numbers, or other symbols on the disk for identification and/or instructing purposes.

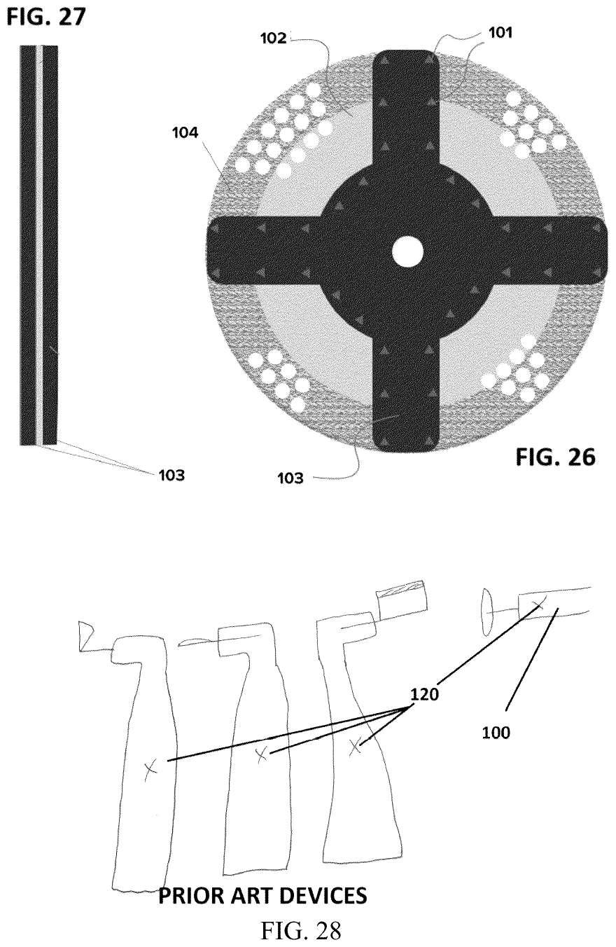

FIG. 26 depicts an alternative design for the disk having a disk base 102 that is made of metal or plastic. This embodiment includes a support structure 103 that is riveted or welded to the base disk 102. Rivets or welds 101 are affixed to the disk to support the structure 103 on the disk. A support metal structure 103 is affixed to the disk to strengthen the disk within each segment, with FIG. 26 showing four segments. The metal support structure 103 can be positioned on one side of the disk or on both sides of the disk. An abrasive coating 104 is also provided adjacent the outer perimeter of the disk. In addition, FIG. 26 depicts a plurality of perforations positioned centrally near the outer periphery of the disk in each segment. As shown, each segment has a different number and arrangement of perforations. FIG. 27 is a cross-sectional side view of the disk showing the base disk material 102 in the central and the support structure 103 surrounding the base disk body 102. As is evident from FIG. 27, in this embodiment, the support structure 103 is positioned on both sides of the disk body 102.

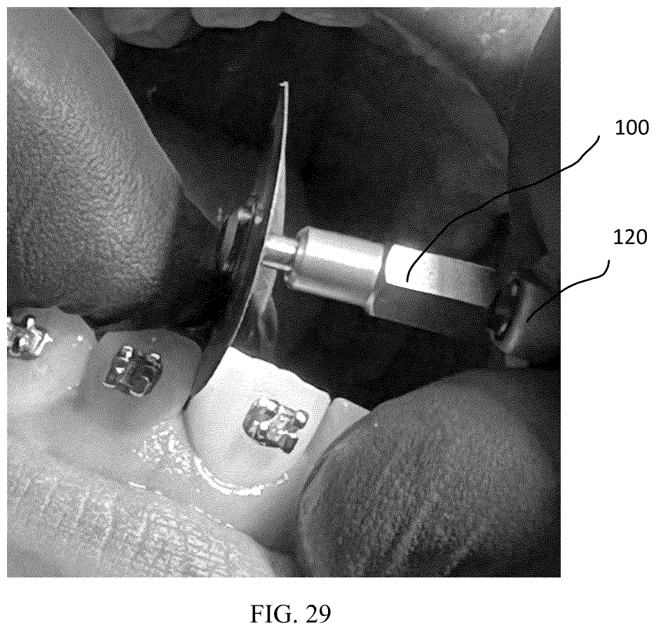

FIG. 28 depicts a sketch of the present handpiece 100 with an attached disk compared to prior art handpieces. FIG. 28 also depicts approximate finger placement on the various tools, designated by reference number 120. Since the tool 100 is a precision instrument, the user has greater control over placement of the disk and overall operation of the tool by positioning the fingers in close proximity to the disk with the present invention. Much like with writing using a writing instrument, to have control of the writing instrument, one places their fingers close to the writing tip. By allowing a user to position their fingers close to the disk in the present invention, it provides for greater accuracy on the part of the dentist, thereby deterring the likelihood of injury to the soft or hard tissues in the mouth.

The present invention uses a straight hand piece 100 that is shorter than prior art handles. Prior art IPR tools use a contra angle handpiece. The main difference between a contra angle handpiece and a straight angle handpiece 100 is that the fingers and hand can be positioned much closer to the cutting edge of the instrument, thereby providing better control for the dentist. With a straight handpiece, the user can put their fingers at the end of the handpiece directly adjacent the cutting edge. With contra angle handpieces, a user holds the tool in the middle of the tool. The sketch in FIG. 28 shows the position of the user's hand during use with the various handpieces currently on the market, signified by an X or reference number 120 in the sketch. As is evident, it is possible to position the hand directly next to the cutting area with the present invention. Thus, the present configuration provides for a different method of use where the user places their fingers directly adjacent to the cutting surface. FIG. 29 depicts an image of the present tool 100 in the mouth of a user, showing the position of the user's fingers 120 directly adjacent the cutting blade in the mouth.

Disks can be sold in kits that include more than one disk having a thickness that ranges from about 0.05 mm to about 0.7 mm. The most common kit may have divisions of about 0.1 mm starting with about 0.1 mm through about 0.5 mm. Disks may be identified or labeled using either color coating, laser markings, or other markings. The laser markings could be cut-out markings. The markings or color coatings can allow the user to identify the thickness of the disk, for example.

Referring to FIGS. 18-23, a sanding tool is also disclosed that includes a handle that has a cylindrical cross-section at an upper end and is generally triangular when viewed in a plan view. The sanding tool is easily held in a user's hand between the thumb and index finger. The handle can range in diameter from 2 mm to 10 mm and have a varying thickness from top to bottom or bottom to top. The handle can have a ball on top and at the end of the handle to help prevent the user's fingers from slipping off when moving the handle. The design of the handle allows the user's fingers to grasp the tool and manipulate it such that the tool can be twisted 360 degrees without needing to drop the tool and pick the tool up so as to access different sides of the sanding surface. The example handle allows the users finger to vertically move the tool Up and Down, or Side to Side, to sand the teeth. The sanding portion of the tool can be either a strip, half circle, or full circle, as shown in FIGS. 18-21, respectively.

As shown in FIGS. 22 and 23, the handle design can also be designed to have a breaking mechanism 96 to allow the user to rotate the disk to different positions and then tighten the sanding disk so it does not rotate during use. The braking system can be a set screw that tightens up against the disk. The set screw may be positioned on the side of the tool or on the face of the tool (as shown in FIG. 23). Alternatively, the set screw can be incorporated into the handle so the user can twist and tighten and loosen the handle to apply pressure to the sanding disk allowing it to move and or prevent it from moving, as shown in FIG. 22. This braking system can be part of the tool that is injection molded at the same time the other parts of the tool are created. Alternatively, a separate metal tightening part may be incorporated into the plastic.

The handle can be fabricated from reusable materials such as plastic or metal. The handle may be assembled and disassembled, allowing the user to change the cutting instrument inside the handle, reducing the costs associated with the sanding tool. The sanding tool may be sectioned in half along the long axis following the blade's diameter. The sanding tool may fit together and be held together with a tightening nut and bolt. In use, the user simply loosens and re-tightens the nut and bolt when changing the sanding portion of the tool.

Sanding tools can be developed in sets, with identifying marks or colors indicating for example abrasive coatings on one of the two surfaces (front and back) or double coated on both the front and back. For further example, a Red, White, and Blue system may be incorporated, with Red indicating Up, White indicating Down, and Blue indicating Up and Down. Other colors, numbers, or markings may alternatively be used.

The present invention is also directed toward a lubricant that can be placed on the surface of either a high-speed cutting instrument, a low-speed instrument, or a hand-held cutting instrument that will lubricate the cutting surfaces of the instruments and the tooth's surface instead of using water alone. The lubricant reduces the friction between dental appliances, such as dental appliances that are designed to be installed on teeth, removed and replaced on teeth, or a combination of both. The lubricant can be used alone or with water. The lubricant can be used with the IPR instrument discussed above or with other instruments. The dental appliances that utilize the lubricant can be either removable or fixed on the teeth, or a combination of both fixed and removable.

The lubricant described herein can be a liquid, gel, powder, spray, foam, an emulsion, or a semi-solid to solid form. The lubricant may have a viscosity that ranges from about 1 to 100,000 centipoise. The viscosity of the formulation at the end of any dilution, dissolution, or breakdown from the form the product is sold prior to being applied to appliances is between 1-200 centipoise, with a preferred range being 1-50 centipoise. The lubricant can be applied to a cutting instrument or to the tooth surface or both by spraying, painting, dripping, pumping, aerosolizing or dipping. The lubricant can be applied to the tooth surface, cutting instrument, or both.

The lubricant can be introduced into the mouth like a mouthwash and swished and either swallowed or expectorated. The lubricant can be introduced into the mouth as a gel directly into the mouth, swished, wiped, or brushed and then either swallowed or expectorated. The lubricant can be introduced into the mouth as a powder that when it comes into contact with saliva, the powder dissolves in the mouth and disperses around the mouth. It can then be either swallowed or expectorated. The lubricant can be introduced into the mouth on a brush and applied to the appliances inside the mouth using the brush to spread the formulation and then is either swallowed or expectorated. The lubricant can be introduced into the mouth as a foam that is swished around appliances in the mouth. The lubricant can be introduced directly at or on the appliances, at points where the appliances interact, contact, or function with other parts of the appliances. The lubricant may be introduced into one appliance in the mouth and then carried to another appliance in the mouth. The lubricant can be introduced into a dental appliance with a syringe at specific friction points between the appliance and the tooth or orthodontic attachments affixed to teeth to facilitate the tooth movement. The formulation can be placed in or on an appliance and then swished and swallowed or expectorated.

The lubricant may be a stand-alone product or can be inserted into other types of products that are used in the mouth, such as, but not limited to, mouth rinses, toothpaste, breath mints, chewing gum, lozenges, troche, sprays, and gummies. The formulation of the lubricant can be produced in different forms, such as gels, pastes, liquids, powder, salve, cream, and gummies.

The formulation can strictly be a lubricant for performing interproximal reduction or other enamel plasty procedures, or can contain other therapeutic properties. These properties include, but are not limited to, enamel strengthening, anti-cavity, anti-tooth sensitivity, analgesic, tooth remineralization, anti-bacterial, anti-plaque, disinfecting, cleaning, anti-halitosis, and whitening. The formulation may include flavoring agents and be packaged in multiple sizes to accommodate users' needs for portability versus larger at home packaging sizes. The formulation can be combined with other known energy sources such as light radiation, vibration, pressure from chewing, and pressure from applying force to the appliances using digits or other objects. Energy from the body in the form of heat can change the properties of the formulation by decreasing viscosity or activating ingredients making them more efficacious.

The formulation will be composed of water ranging from 10-99 percent. The formulation can contain lubricants used in other personal care products formulated for the skin, mouth, hair, or nails. The formulation can contain humectants used in other personal care products formulated for the skin, mouth, hair, or nails. The formulation can contain thickeners used in other personal care products formulated for the skin, mouth, hair, or nails. The formulation can contain natural or artificial flavors used in other personal care products formulated for the skin, mouth, hair, or nails. The formulation can contain emulsifiers used in other personal care products formulated for the skin, mouth, hair, or nails.

The formulation can contain fluorides used in other personal care products formulated for the mouth. The formulation can contain antimicrobials used in other personal care products formulated for the skin, mouth, hair, or nails, including peroxides, chlorhexidine gluconate, antibiotics, antifungals, and antivirals.

The formulation can contain preservatives used in other personal care products formulated for the skin, mouth, hair, or nails. The formulation can contain anti-tooth sensitivity agents used in other personal care products formulated for mouth. The formulation can contain anti-calculus agents used in other personal care products formulated for the mouth. The formulation can contain tooth whitening agents used in other personal care products formulated for the mouth, including chemicals that are or break down into hydrogen peroxide, or other peroxides, releasing oxygen during their breakdown. The formulation can contain binding agents used in other personal care products formulated for the skin, mouth, hair, or nails.

The formulation can contain enamel remineralization agents used in other personal care products formulated for mouth, such as amorphous calcium phosphate, Novamin, and milk derived agents. The formulation can contain emollient agents used in other personal care products formulated for the skin, mouth, hair, or nails. The formulation can contain texture enhancers used in other personal care products formulated for the skin, mouth, hair, or nails.

The formulation can contain natural and artificial colors used in other personal care products formulated for the skin, mouth, hair, or nails. The formulation can contain antioxidant agents used in other personal care products formulated for the skin, mouth, hair, or nails.

The formulation can contain plant extracts agents used in other personal care products formulated for the skin, mouth, hair, or nails. The formulation can contain pigments used in other personal care products formulated the skin, mouth, hair, or nails. The formulation can contain emollient agents used in other personal care products formulated for the skin, mouth, hair, or nails. The fluoridated, interproximal reduction lubricant can contain polishing agents used in toothpastes, with varying abrasives, with different RDA values, including but not limited to sodium bicarb, hydrated silica, etc.

In one embodiment, the lubricant includes active and inactive ingredients. The active ingredient is sodium fluoride. The inactive ingredients include water, glycerin, hydroxyethyl cellulose, propylene glycol, sucralose, mint oil, sodium benzoate and propylparaben.

In one embodiment, a holder for the lubricant can be provided in a kit form with the lubricant being positioned by the user in a holder. The clinician moves the cutting instrument back and forth between the holder and the tooth to apply and then reapply the lubricant between cutting or polishing.

A lubricant or medicament holder can be shaped to receive the cutting instrument in such a way where the holder is shaped to conform to the cutting instrument's shape, so it can be dipped into the well to apply the lubricant. The holder may alternatively be shaped to receive the cutting instrument where the holder is a dappen dish that is disposable and has a cover that when removed gives access to the lubricant and acts like the lubricants packaging. The dappen dish can be part of a complete package that also contains a disposable brush so the user can either paint the lubricant onto the tooth or cutting instrument or dip it into the dual-welled dish/holder form. The lubricant can be expelled from a larger vessel such as a syringe, dropper bottle, or tube into a small well or dappen dish. The holder can be an assembly or tray with one or more dish or well that ranges from one well to a maximum of five wells that reside next to or adjacent one another. The wells can be used by the clinician to dip a brush or the cutting instrument into each well and apply direct solutions to either the tooth or the cutting instrument. Contents of the wells can be a combination of different medicaments or therapeutics or varying types of lubricants or polishers.

In a first example, five wells are utilized as follows:
Well 1: Cleaner
Well 2: Lubricant with Fluoride
Well 3: Course Polish
Well 4: Fine Polish
Well 5: Water

In a second example, four wells are utilized as follows:
Well 1: Lubricant
Well 2: Course Polish
Well 3: Fine Polish
Well 4: Water with fluoride polish

In a third example, three wells are utilized as follows:
Well 1: Lubricant
Well 2: Fine Polish
Well 3: Water with Fluoride

In a fourth example, two wells are utilized as follows:
Well 1: Lubricant
Well 2: Fine Polish with Fluoride

In a fifth example, one well is utilized and contains a lubricant with fluoride.

In a sixth example, one well is utilized and contains a lubricant with fluoride, polish and potassium nitrate.

In a seventh example, three wells are utilized as follows:
Well 1: Lubricant with fluoride
Well 2: Fine Polish
Well 3: Sodium fluoride varnish

In an eighth example, two wells are utilized as follows:
Well 1: Lubricant with fluoride
Well 2: Sodium with fluoride varnish, polish, and potassium nitrate

The dappen dish can be designed to accept the cutting instrument and deliver the lubricant to the cutting surface. The well of the dappen dish can be designed so the lubricant sits in the bottom of the well and as the housing of the cutting instrument is pressed into the dappen dish, it forces the lubricant onto the cutting surface. The interdigitation of the housing and the dappen dish is such that the housing acts as a plunger forcing the lubricant up and around the cutting surface.

The lubricant can be a stand-alone lubricant, or can contain any oral care medicament currently used in dentistry including but not limited to: anti-cavity agents such as fluorides;

enamel building minerals such as calcium and phosphate and or other enamel medicament building agents; anti-sensitivity agents such as fluorides, potassium nitrate and oxalic acid; analgesics such as benzocaine, prilocaine, isocaine, lidocaine, and other anesthetic gels; breath freshening agents, and other agents.

The formulation can contain polishing agents to polish the tooth as the formula lubricates the cutting instrument. A partial list of agents include, but are not limited to:
a. Diamond,
b. Artificial diamond,
c. aluminum hydroxide (Al(OH)₃),
d. calcium carbonate (CaCO₃),
e. calcium hydrogen phosphates,
f. silicas,
g. zeolites,
h. hydroxyapatite (Ca₅(PO₄)₃OH),
i. White mica,
j. Zinc oxide,
k. Silicon carbide or carborundum,
l. Silicon dioxide,
m. Pumice,
n. Sodium silicate,
o. Diatomaceous earth
p. Crystalline silica (quartz),
q. aluminum silicate,
r. carbide compounds,
s. garnet,
t. feldspar,
u. zirconium silicate,
v. zirconium oxide,
w. boron,
x. calcium carbonate,
y. emery, and
z. perlite.

The formulation can contain remineralizing agents from categories:
a. Calcium phosphate,
b. Phosphate,
c. Calcium,
d. Novamin, and
e. Amino acids.

The formulation can contain Fluorides from the following classes:
a. Stannous fluoride,
b. Acidulated fluoride,
c. Sodium fluoride,
d. Sodium mono fluorophosphate, and
e. others used in dentistry not listed.

Antibacterial, antifungal, and antiviral ingredients from categories such as hydrogen peroxides, essential oils, can be incorporated. Triclosan and chlorhexidine can also be incorporated. The formula can also contain anti-sensitivity agents, such as potassium oxalate and potassium nitrate. To deliver the polishing agent to the tooth's surface, and to polish the tooth, an absorbent strip can be either dipped in the polishing agent or the polishing agent can be brushed on or applied from a vessel like a syringe or tube. The polishing strip can be fabricated from fabric, leather, or synthetic fibers, and/or be solid. The polishing strip may be perforated to add additional retentive area for the lubricant. The polishing strip can be long enough to allow the clinician to hold it using two hands and move it back and forth to polish the tooth. The polishing strip can be affixed tightly between a horseshoe shaped member fabricated from either metal or plastic, disposable or multi-use, which allows the clinician to polish the surfaces after applying the polish.

Various wells can be added to a sanding strip's handle. A port can be added to the handle of sanding strip holders in which a lubricant can be injected at the top of the handle and drip onto the cutting surface. The port can be constructed in a range of sizes to hold various amounts of lubrication. A well can be added at the top of the cutting instrument where it enters the handle to allow the clinician to inject an amount of lubricant into the well, which can then drip onto the cutting surfaces.

A dual use lubricant can be utilized that the clinician uses to perform the IPR Procedure and is then applied to the cut teeth and allowed to remain for a period of time so that it can soak into the ename; or, the same package (say a syringe) can be used during the IPR procedure and the remaining lubricant with for example fluoride is given to the patient to apply at home to strengthen the cut enamel. This lubricant can be used before, during, and after IPR treatment.

A pen or marker, like a sharpie marker with ink that is non-toxic can be used to mark the location of where the doctor performed IPR so the patient can know when they go home where to apply the medicated IPR gel to remineralize the freshly cut enamel.

A manufacturer of the orthodontic aligners or other dental appliances can mark the aligner with a marking ink or other markings, such as a dimple or hole in the plastic so the patient knows the location of where the doctor wishes the patient to apply therapeutics to the device to deliver the therapeutic to the desired site of the teeth or the gums. An X or period, for example, can be used to mark the spot for lubrication deposition. The markings can be applied and treatment planned based on the location of gum disease or where doctors have cut enamel and where the doctor wishes the patient to apply medicament.

A manufacturer of the aligners can create recesses where the IPR was performed in the aligner and preload the aligners with the fluoride gel so that after the doctor performs the IPR, they may provide the patient with an aligner that has grooves positioned adjacent the affected area where fluoride gel or other medicaments can be applied. The fluoride gel may be activated by saliva to release and remineralize the teeth.

Any number of different additional inactive ingredients may be added to the formulation, including those shown in Table 1 below.

TABLE 1

| | | |
|---|---|---|
| Acrylate Copolymer | Fragrance Coconut Lime | Pigment Blend Bare Beige |
| | Verbena | |
| ActiCap ®s Brightening | Fragrance Coral Reef | Pigment Blend Bare Neutral |
| ActiCaps ® Argireline | Fragrance Lemon Verbena | Pigment Blend Caramel |
| ActiCaps ® Eyes | Fragrance Mandarin-Berry | Pigment Blend Earth Brown |
| ActiCaps ® Retinol | Fragrance Mangosteen | Pigment Blend Honey Beige |
| AHA Fruit Acids | Fragrance Natural Rose | Pigment Blend Natural Buff |
| Ajurana EyeContour, ECOCERT | Fragrance Neroli | Pigment Blend Sample Kit |
| Approved | | |
| Algae Extract | Fragrance Pineapple Lily | Pigment Blend Warm Beige |
| Algae Extract & Hyaluronate | Fragrance Pink Grapefruit | Pigment Blue No. 1 FD&C Lake |
| | Passion Fruit | |
| Algae Extract, USDA Certified | Glyceryl Stearate Citrate | Pigment Carbon Black |
| Organic | | |
| Alkyl polyglycoside | Glyceryl Stearate SE | Pigment Chromium Oxide Green |
| Allantoin | Glycol Distearate | Pigment Iron Oxide Black |
| Almond oil Emollients | Glycol Stearate IP | Pigment Iron Oxide Black |
| | | (Liquid) |
| Almond Oil | Glycolic Acid | Pigment Iron Oxide Brown |
| Almond Oil, USDA Certified | Glycoproteins | Pigment Iron Oxide Brown |
| Organic | | (Liquid) |
| Aloe barbadensis leaf juice powder | glycerine oral demulcent | Pigment Iron Oxide Red |
| (Aloe vera) | | |
| Aloe Barbadensis Powder | Goji Berry Extract, ECOCERT | Pigment Iron Oxide Red |
| | Approved | (Liquid) |
| Aloe Vera 10x Concentrate | Goldenseal Extract | Pigment Iron Oxide Yellow |
| Aloe Vera Palmitate | Gotu Kola Extract | Pigment Iron Oxide Yellow |
| | | (Liquid) |
| Aloe Vera Powder | Grape Seed Extract | Pigment Red No. 28 D&C Lake |
| Aloe Vera Pure Juice | grapefruit seed extract | Pigment Red No. 40 FD&C |
| | preservative | Lake |
| Alovera Alpha Olefin Sulfonate | Grapeseed Oil | Pigment Red No. 6 D&C Lake |
| Alpha-Arbutin | Green Tea Butter | Pigment Red No. 6 D&C Lake |
| | | (Liquid) |
| Aluminum Chlorohydrate | Green Tea Extract, ECOCERT | Pigment Red No. 7 D&C Lake |
| | Approved | |
| Amaranthus Seed Extract | Guar Gum (cationic) | Pigment Red No. 7 D&C Lake |
| | | (Liquid) |
| ammonium lauryl sulfate | Gum Arabic (prehydrated) | Pigment Ultramarine Blue |
| anionic surfactants | HairFix Powder | Pigment Ultramarine Pink |
| Amodimethicone | HairFix XH Maltodextrin | Pigment Violet No. 2 External |
| | | D&C |
| Apricot Kernel Oil | HE-Cellulose, Modified | Pigment Yellow No. 5 FD&C |
| | | Lake |
| argan oil Emollients | Hectorite Gel SOFT | Plankton Extract |
| Argan Oil, USDA Certified | Helianthus annuus (Sunflower) | Poloxamer 338 |
| Organic | seed oil | |
| Argireline NP | Hemp Seed Oil | Poloxamer 407 foaming agent |
| Arnica Extract | Hexanediol CG | polyacrylic acid |
| AromaBenzoic Acid | Homosalate | Polyamide 3 |
| Arrowroot Starch | Honey Extract, ECOCERT | Polycarbophil |
| | Approved | |
| Artichoke Leaf Extract | Honeysuckle Blend | Polyethylene |
| Avena Sativa (Oat) Kernel Flour | Horse Chestnut Extract | Polyglucose |
| Avobenzone | HP Starch | Polyglycerol (Diglycerol) |
| | | emulsifier |
| Avocado Butter | hyaluronic acid Humectants | Polyglyceryl Oleate |
| Avocado Oil | Hyaluronic Acid | Polyglycitol |
| Avocado Powder | Hyaluronic Acid SLMW | Polyhydroxystearic Acid |
| Bacillus Ferment | HydroComplex | Polyisobutene 1200 |
| Bamboo Extract | Hydrogenated Palm Oil | Polyisobutene 250 |
| | Glyceride | |
| Bamboo Extract in Safflower Oil | hydrogenated starch | Polyisobutene 800 |
| Bamboo Stem Powder | hydrolysate | Polymethylsilsesquioxane |
| Baobab Oil, USDA Certified | humectant and sweetener | Polyquaternium 10 |
| Organic | | |
| Baobab Protein, Hydrolyzed | hydrolyzed proteins | Polyquaternium 15 |
| Base, Antioxidant Cream | Humectants | Polyquaternium-7 |
| Base, Antioxidant Lipstick | Hydroxyethylcellulose | Polysorbate 20 |
| Base, Balanced Cream | Hydroxypropyl Guar | Polysorbate 60 |
| Base, Body Butter Creme | Hydroxypropyl Methylcellulose | Polysorbate 80 |
| Base, Botanical Gel | ICE Alginate | Potassium acesulfame sweetener |
| Base, Botanical Toner | ICE Blend | Potassium sorbate preservative |
| Base, Clay Mask | ICE Conditioner | Potassium Sorbate |
| Base, Cleansing Cream | ICE Hair Restore | potassium thiocyanate |
| Base, Cleansing Gel | ICE HairGel | Propanediol 1,3 |
| Base, Concentrated Cream | ICE Silicone | Propylparaben humectant and |
| | | propylene glycol emulsifier |
| Base, Cream Mask | ICE Sunflower | propylene glycol Humectants |
| Base, Fruit Acid Cream | Irish Moss Extract | Propylene Glycol |
| Base, Herbal Cream | Iso-Dimethicone Copolymer | propylparaben preservative |
| Base, Herbal Hair & Body Wash | Isododecane | Provitamin B5 (d-panthenol) |
| Base, Hydro Spray | Isoeicosane | Provitamin B5 Powder (dl- |
| | | panthenol) |
| LotionBase, Matte Liquid Lipstick | Isohexadecane | Prunus dulcis (Sweet almond oil) |
| Base, Mineral | IsoLanolin | Rhubarb Root Extract |
| Base, Mineral Powder | Isopropyl Myristate | Rice Bran Beads |
| Base, Moisturizing Lipstick | Isopropyl Palmitate | Rice Quat |
| Base, Natural Eye Shadow | isopropyl stearate | Rice Starch |
| Base, Natural Lip Balm | Jojoba Castor Beads | Rosa damascene flower oil |
| | | (Rose) |
| Base, Premium Cream | Jojoba Gel | Rose Flower Extract |
| Base, Serum | jojoba oil | Rose Hip Oil |
| Base, Silky Cream | Jojoba Oil, USDA Certified | Rose Hydrosol |
| | Organic | |
| Base, Strands Pomade | Jojoba Pearls | Rosemary extract wound healing |
| Base, Vitamin C Cream | Jojoba Protein HP, Hydrolyzed | Rosemary Leaf Extract |
| Base, Vitamin Serum | Kakadu Plum Extract | Rosemary Leaf Extract, USDA |
| | | Certified Organic |
| Bees Wax | Kaolin | Saccharomyces Ferment, USDA |
| | | Certified Organic |
| Behentrimonium | Kaolin Clay, Green | Sage Extract |
| Cationic surfactants - Quats | Kaolin Clay, Red | Salicylic Acid |
| Behentrimonium chloride | Kaolin Clay, Yellow | Salicylic Acid Solution |
| Benzoic acid | Keratin Protein, Hydrolyzed | Sea Buckthorn Extract |
| Benzophenone-4 | Knotgrass Flavonoids | Sea Fennel Extract |
| Benzyl Alcohol | Kojic Acid | Sea Kelp Extract |
| Benzylalcohol-DHA | Lactic Acid | Sea Kelp Extract, USDA |
| | | Certified Organic |
| Beta Glucan | Lacto-Ceramide | SeaFerment SA |
| BHT | Lactoferrin | Sebum-REG |
| Bismuth Oxychloride | Lactoperoxidase | Sesame Seed Oil, USDA |
| | | Certified Organic |
| Brassica Alcohol | lanolin alcohol | Shea Butter Glycerides |
| Brassica Glycerides | Lanolin Wax | Shea butter oil |
| butylene glycol humectant | Lauramide diethanolamine | Silica |
| | (DEA) nonionic surfactant | |
| Butylene Glycol | Lauramine oxide nonionic | Silica Dimethyl Silylate |
| | surfactant | |
| Butyrospermum parkii (Shea | Laureth sulfate (sodium lauryl | Silicone dioxide (inactive) |
| butter) | ethersulfate or SLES) anionic | |
| | surfactants | |
| C12-15 Alkyl Benzoate | Laureth-3 | Silicone Gel |
| calcium glycerphosphate anticaries | | |
| Calcium Lactate | Lauryl betaine nonionic plant | Silicone Resin |
| | based | |
| Calcium phosphate | Lauryl glucoside surfactant | Silk Protein, Hydrolyzed |
| Calendula Extract | lauryl laurate Emollient Esters | SkinFirm Dipeptide |
| Calendula Officinalis Flower | Lavender Essential Oil | SkinLift DPHP |
| Extract | | |
| Camelina Oil | Lavender Hydrosol | SkinRenewal Complex |
| Candelilla Wax | Lecithin Powder, USDA | Skin Tight AP |
| | Certified Organic | |
| canola oil | Lecithin, USDA Certified | Skin Tight C-Root |
| | Organic | |
| Caprylhydroxamic Acid GG | Licorice Extract | SkinWhite Ascutin |
| Caprylic Acid | Licorice Extract in Safflower | Skin White BLE |
| | Oil | |
| ComboCaprylyl Glycol EHG | Licorice Extract, USDA | Skin White Herb, ECOCERT |
| | Certified Organic | Approved |
| Caramel Colorant thickener | Lingonberry Stem Cells | Skin White MSH |
| emulsifier gelling agent | | |
| Carbomer | Linum usitatissimum (Flax) | Skin White MSH Fluid |
| | seed extract | |
| Carbomer 940 | Lip Balm Base, Natural | Skin White Punarnava |
| Carbomer 980 QD | LiPeptide | Skin White Starflower |
| Carbomer Homopolymer Type B | Lipstick Base, Antioxidant | SM Cocoyl Taurate |
| Carboxymethylcellulose | Lipstick Base, Matte Liquid | SM Cocoyl Taurate Powder |
| Carnauba Wax | Lipstick Base, Moisturizing | SN-KE Peptide |
| Carrageenan thickener, stabilizer | Liquid Minerals (Nigari) | Soapwort Extract |
| Carrot Cells | Lupine Protein, Hydrolyzed | sodium benzoate preservative |
| Carrot Oil & Beta-Carotene | Lychee Extract | Sodium Benzoate |
| Castile Soap | Lysozyme | sodium bicarbonate |
| Castor Oil | Macadamia Nut Oil | Sodium Carboxymethylcellulose |
| | | lubricant |
| Castor Wax | Magnesium Aluminum Silicate | Sodium chloride |
| Cellulose Gum thickener stabilizer | Magnesium Stearate | Sodium Citrate |
| Cera alba (Bees wax) | Mallow Extract, ECOCERT | Sodium Cocoyl Isethionate |
| | Approved | (SCI) |
| Ceramide Complex | Maltodextrin | sodium diphosphate |
| Ceratonia siliqua (Locust bean | Mango Butter, USDA Certified | Sodium Gluconate buffer |
| gum) | Organic | preservative, flavorant anionic |
| | | surfactants |
| Ceteareth-20 | Marrubium Extract, ECOCERT | Sodium hyaluronate |
| | Approved | |
| Ceteareth-25 | Marshmallow Root Extract, | Sodium Hydroxide amphoteric |
| | ECOCERT Approved | surfactants |
| Cetearyl Alcohol | Marula TetradecaneMC Eye | Sodium lauraminopropionate |
| | Lash Serum | |
| Cetrimonium Chloride | Meadowfoam Seed Oil | Sodium Lauroyl Methyl |
| | | Isethionate (SLMI) anionic |
| | | surfactants |
| Cetyl Alcohol | Menthol Crystals | Sodium lauryl sulfate (sodium |
| | | dodecyl sulfate, SLS, or |
| | | SDS) anionic surfactants |
| Cetyl palmitate Emollient Esters | Methyl Gluceth-10 preservative, | Sodium myreth sulfate anionic |
| | antifungal | surfactants |
| Cetyl Palmitate | methyl paraben | Sodium pca Humectants |
| Cetylpyridinium chloride | Methylcellulose | Sodium Phosphate pH adjuster |
| anticeptic/preservative | | |
| Chamomile Extract | Mica Beige | Sodium polyacrylate |
| Chamomilla Recutita (Matricaria) | Mica Blackstar Red | Sodium Saccharin sweetener |
| Flower Extract | | |
| Chaparral Extract | Mica Bordeaux | Sodium Stearate |
| Charcoal Powder | Mica Bronze | sodium sulfite |
| Chlorhexidine Gluconate | Mica Carmine Red | Sorbic Acid |
| Cholecalciferol | Mica Cinnamon | Sorbitan Oleate |
| Cholesterol NF preservative, metal | Mica Diamond Cluster | Sorbitan Stearate |
| chelator | | |
| Citric Acid | Mica Diamond Sparkle | Sorbitol Humectants |
| Citronella Oil | Mica Fine Silver | Soy-Rice Peptides |
| Citrus Combo | Mica Gold | Spearmint |
| Cleansing Blend WF | Mica Interference Copper | Squalane |
| Cocamide DEA nonionic surfactant | Mica Interference Green | Squalane Light Cationic |
| amphoteric surfactants | | surfactants-stearalkonium |
| | | chloride Quats |
| Cocamidopropyl betaine | Mica Interference Violet | Stearamine oxide nonionic |
| | | surfactant |
| Cocamidopropyl Hydroxysultaine | Mica Light Blue | Stearic Acid |
| Cocamidopropylamine Oxide | Mica Luster Black | Stearyl Alcohol |
| Coco Betaine | Mica Magenta | Stearyl Palmitate |
| Coco Glucose | Mica Majestic Green | Sucralose sweetener |
| Cocoa Butter Wafers Deodorized, | Mica Patina Silver | Sucrose Cocoate |
| USDA | | |
| Certified Organic | Mica Pearl White | Sulfosuccinate |
| Cocoa Butter, USDA | | |
| Certified Organic amphoteric | Mica Pink Camel | Sulphur Mud |
| surfactants | | |
| Cocoamphopropionate | Mica Powder | Summer Lilac & Thyme Extract, |
| | | ECOCERT Approved |
| Coconut oil Emollients | Mica Purple Sky | Sunflower oil emollient |
| Coconut Water | Mica Red | Sunflower Oil, USDA Certified |
| | | Organic |
| Coenzyme Q10 (with Vit. E & C) | Mica Sand Gold | Sunflower Petal Extract |
| Collagen Protein, Hydrolyzed | Mica Sappan Red | Sunflower Solubilizer |
| Colloidal Oatmeal | Mica Sparkle Gold | Sunflower Wax |
| Conditioner SD thickener, | Mica Spheres | Synthetic Wax |
| lubricant, film former | | |
| Copovidone | Mica Sugar Blush | T-Resveratrol Fluid |
| Cranberry Seed Oil | Mica Walnut Brown | Talc |
| CreamMaker ® ANIO | Microcrystalline Wax | Tapioca Starch |
| CreamMaker ® BLEND | Microcrystalline Wax Pastilles | Tara Gum Gel |
| CreamMaker ® CA-20 | Milk Protein, Hydrolyzed, | Tea Tree Essential Oil |
| | ECOCERT Approved | |
| CreamMaker ® CAT | Mineral Base | Teprenone |
| CreamMaker ® FLUID | Mineral Oil | Theobroma cacao (Cocoa) seed |
| | | butter |
| CreamMaker ® Green Coffee | Moisture REG | Titanium Dioxide (micronized) |
| CreamMaker ® MIX | monobasic sodium phosphate | Titanium Dioxide for Makeup |
| CreamMaker ® Moringa | Mulberry Root Extract | Titanium Dioxide in Aqua |
| CreamMaker ® Silicone | Multi Herb Oily Skin | Titanium Dioxide in Oil hydrates |
| | | skin and preservative |
| CreamMaker ® WAX | Myristic Acid | Tocopherol (Vitamin E) |
| Cucumber Fruit Extract | Myristyl myristate Emollient | Tocopheryl acetate |
| | Esters | |
| Cucumber Fruit Extract in | Myristyl Myristate | Tomato Lycopene |
| Safflower Oil | | |
| Cucumber Fruit Extract, USDA | Natural Bisabolol | Tribehenin |
| Certified Organic | | |
| Cyamopsis tetragonolobus (Guar | Natural Enzymes | Triethanolamine |
| gum) | | |
| Cyclomethicone | Natural Gel Wax | Triglyceride |
| Cyclomethicone Dead Sea Mud | Neroli Hydrosol, USDA | Triglyceride Blend |
| | Certified Organic | |
| Decyl glucoside | Niacinamide | Trihydroxystearin |
| Decyl Glucoside Sodium Lauroyl | Nonionic plant-based surfactant | Tripeptide-5 |
| nonionic plant-based surfactant | | |
| Lactylate | | |
| DeoConcentrate Household | Oat Amino Acids | Trisodium HEDTA |
| DeoConcentrate Personal Care | Oat Emollient, ECOCERT | Urea |
| | Approved | |
| Desert Fruit Blend | Oatmeal Extract | Vaccinium Macrocarpon |
| dibasic sodium phosphate | | (Cranberry) Fruit Extract |
| dicetyldimonium chloride | | |
| Dihydroxyacetone Cationic | Octocrylene nonionic plant- | Vitamin A (retinyl palmitate) |
| surfactants - Quats | based surfactant | |
| Diisooctyl Succinate | Octyl glucoside | Vitamin A Liposomes (retinol) |
| Dimethicone | Octyldodecanol | Vitamin A Microcaps (retinol) |
| Dimethicone 1.25% | olive oil Emollients | Vitamin B3 |
| Dimethicone 500 | OM-Cinnamate | Vitamin C (3-glyceryl ascorbate) |
| Dimethicone Fluid | Orange Peel Butter | Vitamin C (L-ascorbic acid) |
| Dimethicone Satin | Orange Stem Cells | Vitamin C (L-ascorbyl |
| | | palmitate) |
| Disodium Phosphate pH adjuster | Orange-Lemongrass Blend | Vitamin C (magnesium ascorbyl |
| | | phosphate) |
| Distearyldimonium Chloride | Orchid Stem Cells | Vitamin C (sodium ascorbyl |
| | | phosphate) |
| DMDM Hydantoin | Oxybenzone | Vitamin C (tetrahexyldecyl |
| | | ascorbate) |
| Edelweiss Extract, ECOCERT | Ozokerite Wax | Vitamin C in Silicone (20% L- |
| Approved | | ascorbic acid) |
| EDTA | Palmitoyl Isoleucine | Vitamin E (dl-alpha tocopherol) |
| Elastin Protein, Hydrolyzed | Palmitoyl Peptide Complex | Vitamin E (dl-alpha tocopheryl |
| | | acetate) |
| Erythrulose | Palmitoyl Tripeptide-38 | VitaminMIXI |
| Ethoxydiglycol | Paraben-DU | VollLip |
| Ethyl p-hydroxybenzoate | Passiflora Incarnata Flower | Walnut Shell Powder |
| antifungal preservative | Extract | |
| Ethylhexyl Palmitate | Pea Extract, ECOCERT | Water |
| | Approved | |
| Eye Lash Hexa Peptide | Pearl Powder emulsifier, | Watermelon Extract |
| | surfactant, cleansing, | |
| | solubilizing peg 60 | |
| Eye Lash Penta Peptide | PEG-100 stearate | Wheat Protein, Hydrolyzed |
| EyeComplex 4 | PEG-150 Distearate | whey protein skin softening |
| Fatty alcohols nonionic surfactant | PEG-40 Hydrogenated Castor | White Tea Extract |
| | Oil | |
| Fatty alkanolamides nonionic | PEG-60 Hydrogenated Castor | Willow Bark Extract |
| surfactant | Oil | |
| Ferulic Acid, Natural | PEG-7 Glyceryl Cocoate | Willow Bark Extract SA |
| Flavor Cream Soda | PEG-8 Beeswax | Willow Bark Extract, USDA |
| | | Certified Organic |
| Flavor Creme-de-Coco | PEG-8 Dimethicone | Witch Hazel Extract |
| Flavor Guava-Pineapple | Pentylene Glycol | Wrinkle Blur |
| Flavor Vanilla | Pentylene Glycol BIO | Xanthan Gum thickener |
| | | stabilizer |
| flavors natural and artificial | Peppermint | Xanthan Gum (prehydrated) |
| | | sugar alcohol sweetener |
| Follicle Booster | Peppermint Oil, USDA | Xylitol |
| | Certified Organic | |
| Fragrance | Petrolatum | Yogurt Filtrate |
| Fragrance Blood Orange | Petroleum Jelly (Petrolatum) | Zea mays (corn) oil |
| Fragrance Citrus Punch | Phenoxyethanol SA | zinc gluconate |
| Fragrance Clean Linen | Phenoxyethanol | Zinc Oxide |
| Fragrance Coconut | Phenylpropanol EHG | Zinc Oxide (micronized & |
| | | coated) |

The term “substantially,” if used herein, is a term of estimation.

While various features are presented above, it should be understood that the features may be used singly or in any combination thereof. Further, it should be understood that variations and modifications may occur to those skilled in the art to which the claimed examples pertain. The examples described herein are exemplary. The disclosure may enable those skilled in the art to make and use alternative designs having alternative elements that likewise correspond to the elements recited in the claims. The intended scope may thus include other examples that do not differ or that insubstantially differ from the literal language of the claims. The scope of the disclosure is accordingly defined as set forth in the appended claims.

What has been described above includes examples of one or more embodiments. It is, of course, not possible to describe every conceivable modification and alteration of the above devices or methodologies for purposes of describing the aforementioned aspects, but one of ordinary skill in the art can recognize that many further modifications and permutations of various aspects are possible. Accordingly, the aspects described are intended to embrace all such alterations, modifications, and variations that fall within the spirit and scope of the appended claims. Furthermore, to the extent that the term “includes” is used in either the details description or the claims, such term is intended to be inclusive in a manner similar to the term “comprising” as “comprising” is interpreted when employed as a transitional word in a claim. The term “consisting essentially,” if used herein, means the specified materials or steps and those that do not materially affect the basic and novel characteristics of the material or method. All percentages and averages are by weight unless the context indicates otherwise. If not specified above, the properties mentioned herein may be determined by applicable ASTM standards, or if an ASTM standard does not exist for the property, the most commonly used standard known by those of skill in the art may be used. The articles “a,” “an,” and “the,” should be interpreted to mean “one or more” unless the context indicates the contrary.

What is claimed is:

1. A tool for interproximal reduction comprising:
a handpiece having a motor coupled to a coupling member, where said handpiece creates an oscillating motion with a preferred degree of oscillation of 180 degrees or less; or a handpiece having a motor coupled to a coupling member, where said motor creates an oscillating motion with a preferred degree of oscillation of 180 degrees or less;
a substantially solid disk-shaped member having a front face and a rear face and defining two or more segments on each face that are the same as or different from one another, with each segment having different properties comprising one or more of perforations, cutting surfaces, dimples, and abrasives, said disk-shaped member having a centrally disposed aperture for coupling to the handpiece with the coupling member, wherein the handpiece is configured to permit a user to position their fingers directly adjacent the disk-shaped member during operation.

2. The tool of claim 1, wherein the properties only partially cover one or more of the front or rear face of the disk.

3. The tool of claim 1, wherein at least one of the properties is a radially extending cutting member extending outwardly from the aperture to outer circumference of the disk-shaped member; and the properties is an abrasive area and the abrasive area is spaced from the aperture.

4. The tool of claim 3, wherein the abrasive area is shaped as a ring of abrasive that is positioned directly adjacent an outer periphery of the disk in one or more of the segments.

5. The tool of claim 1, wherein one or more of the properties comprises one or more perforations that extend through the substantially solid disk-member; and
the properties comprise a plurality of dimples extending outwardly from the face of the disk.

6. The tool of claim 5, wherein the one or more perforations comprises a plurality of perforations arranged in a pattern, with the perforations being positioned closer to the outer periphery of the disk than to the centrally disposed aperture.

7. The tool of claim 1, wherein the disk is made of plastic or metal and has a thickness ranging from about 0.03 mm to about 0.5 mm.

8. The tool of claim 1, wherein the degree of oscillation is between about 1 degree and about 45 degrees.

9. The tool of claim 1, further comprising a support structure coupled to the disk-shaped member.

10. The A cutting member for use with an interproximal reduction handpiece comprising:
a substantially solid disk-shaped member having a front face and a rear face and defining two or more segments that are different from one another, with each segment having different properties comprising one or more of perforations, cutting surfaces, and abrasives, said disk-shaped member having a centrally disposed aperture for coupling to the handpiece using the coupling member, wherein the properties only partially cover one or more of the front or rear face of the disk; or
a substantially solid wedge-shaped member forming a partial circle, with the partial circle being ¼ or less of a circle and having a front face and a rear face, with the wedge-shaped member having different properties comprising one or more of perforations, cutting surfaces, and abrasives, said wedge-shaped member having an aperture defined at a pointed end of the wedge-shaped member for coupling to the handpiece, wherein the properties only partially cover one or more of the front or rear face of the wedge-shaped member.

11. The cutting member of claim 10, wherein one of the properties is a radially extending cutting member extending outwardly from the aperture to outer circumference of the disk-shaped member;
one of the properties is an abrasive area and the abrasive area is spaced from the aperture; or
one of the properties comprises one or more perforations that extend through the substantially solid disk-member.

12. The cutting member of claim 11, wherein the abrasive area is shaped as a ring or a partial ring of abrasive that is positioned directly adjacent an outer periphery of the disk in one or more of the segments.

13. The tool of claim 11, wherein the one or more perforations comprises a plurality of perforations arranged in a pattern, with the perforations being positioned closer to the outer periphery of the disk than to the centrally disposed aperture.

14. The tool of claim 10, wherein the disk is made of plastic or metal and has a thickness ranging from about 0.03 mm to about 0.5 mm.

15. The tool of claim 10, further comprising a support structure coupled to the disk-shaped member.

16. A method for using the tool of claim 1 comprising;
operating the tool between two teeth of a patient to cut one or more of the teeth and providing a lubricant at the space between the two teeth during operation of the tool, wherein the lubricant is water-based and reduces friction at the cutting surface of the teeth, and serves as a carrier for fluoride or other medicaments during operation to assist in remineralizing the enamel of the teeth; and the lubricant assists in sliding a cutting member of the tool between the two teeth prior to operation of the tool.

17. A lubricant for use in IPR procedures comprising:
a water-based lubricant that reduces friction, said lubricant being a carrier for ingredients that permit the lubrication of cutting during IPR and that lubricates the connection between the teeth to permit a cutting member to slide between the teeth, said lubricant having a viscosity that is different from water alone.

18. The lubricant of claim 17, wherein the lubricant has a viscosity that permits it to stick to the cutting member and to the teeth prior to and during a cutting operation during IPR.

19. The lubricant of claim 18, wherein the lubricant further comprises fluoride and one or more medicaments that aid in remineralizing the enamel of the teeth, or that includes polishing agents to permit easier cutting of the teeth during IPR procedure.

20. The lubricant of claim 17, wherein the lubricant includes active and inactive ingredients, with the active ingredient being sodium fluoride and inactive ingredients including water, glycerin, hydroxyethyl cellulose, propylene glycol, sucralose, mint oil, sodium benzoate and propylparaben.

21. The cutting member of claim 1, wherein the member is coated in whole or in part with a naturally lubricating surface treatment; and the abrasive material coupled to the cutting member is coated with a naturally lubricating surface treatment to enhance the ability of the abrasive to be positioned between teeth and to enhance the abrasive's efficiency.
